(19) United States
(12) Patent Application Publication
(10) Pub. No.: US 2002/0081570 A1
(43) Pub. Date: Jun. 27, 2002

(54) System to detect protein-protein interactions

(76) Inventors: Jack Lilien, Iowa City, IA (US); Lisa Elferink, Grosse Pointe Park, MI (US); Janne Balsamo, Iowa City, IA (US); John Kamholz, Ann Arbor, MI (US)

Correspondence Address: VENABLE, BAETJER, HOWARD AND CIVILETTI, LLP, P.O. BOX 34385, WASHINGTON, DC, 20043-9998, US

(21) Appl. No.: 09/836,865

(22) Filed: Apr. 18, 2001

Related U.S. Application Data
(60) Provisional application No. 60/198,122, filed on Apr. 18, 2000.

(51) Int. Cl. C12Q 1/70; G01N 33/53

(52) U.S. Cl. 435/5; 435/7.1

(57) ABSTRACT

A method for screening protein-protein interactions that is rapid, easy and generally applicable to a wide array of such interactions is disclosed. This method, an adaptation and combination of certain existing approaches, the T7 phage display libraries and target epitope arrays made, for example, by simultaneous synthesis overlapping peptides of known sequence. These methods provide for high throughput screening that can identify the particular amino acids or domains or epitopes that are of primary importance in the binding interactions between two protein partners.

### BACKGROUND OF THE INVENTION

1. Field of the Invention

The invention in the field of proteomics relates to novel methods for identifying proteins, or peptide domains thereof, that bind to and interact with selected target epitopes, primarily of other peptides. The method combines the technique of phage display libraries in bacteriophage T7 with target epitope arrays generated, for example, by simultaneous synthesis of overlapping peptides of known sequence.

2. Description of the Background Art

Proteomics is the study of proteins, whereas genomics is the study of DNA and the processes which lead to the creation of proteins. When used in combination, these two approaches to the study of gene expression enable researchers to analyze regulation at many levels. For example, when a cell receives a signal, such as a growth factor, it responds first at the protein level. Cell surface protein receptors are activated and modified. In addition, transmission of information from the activated receptor to the nucleus often involves physical movement of proteins. These activities can be detected and analyzed using proteomic technologies.

One of the key developments in proteomics was the development of 2-dimensional (2D) gel electrophoresis, and subsequent improvements in the technology including commercially available standardized gels and reagents which deliver reproducible results. Such proteomics technology platforms have been improved in concert with gene expression microarrays and genomic databases, leading to the commercially development of protein expression and sequence databases. For example, Incyte's LifeProt™ database contains annotated protein expression data for numerous tissues. Researchers can investigate 2D gel images on screen, looking at identified proteins, obtain amino acid sequence data or link to matching expressed sequence tags (ESTs) in human gene sequence databases.

As more is learned, the path from genome to system seems harder. The simple view of protein synthesis (as might be found in a high school textbook) explains that DNA is transcribed into a corresponding sequence of mRNA, which is then read by the ribosome (translated) to create an amino acid chain (sequence) which folds up into a three-dimensional shape and becomes a functional protein, which goes to some part of the cell (or elsewhere in the body) to perform its particular role. It was long believed that one gene was responsible for encoding one polypeptide, so that the number of genes in a human should be equal to or greater than the number of distinct proteins we produce. It is also well-known that things are not quite this simple; confounding factors between gene and protein function seem to mount with every discovery.

“Between the chromosome and the ribosome,” RNA can be spliced and recombined, meaning that one gene can encode more than one protein. While this phenomenon has been known for many years, the amount of RNA variation that derives from a single gene was not realized until relatively recently. RNA “editing” occurring through a series of enzymatic reactions can create as many as 50 variant RNA chains from a single gene. These edited variants can be difficult to track by genomic methods because it is difficult to predict the number of splice variants. Editing may go undetected as there are to few genomic sequences compared to RNA sequences.

Protein diversity is enlarged further by posttranslational modification of amino acids by different (chemical) functional groups, e.g., phosphorylation and dephosphorylation, glycosylation and deglycosylation, which could change the function as well as the targeting of the protein. Some proteins are created in an inactive form, then enzymatically cleaved, converting them to a new and active form. In recent years, the role of “chaperoning,” a type of protein that assists folding of other proteins in the cell, has been discovered, adding one more factor to the final shape and function. For reasons not fully understood, the mere time and place of protein synthesis can affect function, independent of structural protein/protein interactions or glycosylation patterns. The reasons remain obscure. Different amino acid sequences can actually fold into the same shape—at least in active regions—and therefore take on identical functions. Examples of this are chymotrypsin and subtilisin—independently evolved serine proteases with identical active regions and functions. More important for the present invention, proteins interact with each other and with other organic molecules to form pathways.

The genomics industry is based on the idea that sequence information can be used to predict real things about complex biological organisms and allow discovery of targets for new therapies, even therapies customized to an individual. Despite the confounding factors (discussed above) between DNA sequence and phenotype, this gap will surely be bridged. But to reach that point, new tools are needed. Proteomics is emerging as a high-throughput technology that allows researchers to take a step further down the “function” chain by studying actual proteins post-synthesis and determining their amino acid sequences. But even this kind of information only goes so far by itself if a given amino acid sequence folds differently under different circumstances—proteomics will not easily be able to identify all those changes. Such complications make protein-protein interactions even more difficult to predict. The present invention provides one tool to overcome such hurdles.

How many proteins do we have? From the one gene-one protein days, some have estimated on the order of 10⁵ different proteins in each mammalian organism. That estimate has risen to 10⁵ genes capable of encoding 10⁶ or more protein forms, though information gained from the sequencing of the human genome has led to an estimate of about 4×10⁴ genes encoding at least 10⁶ proteins. A single gene could, based on some of these estimates, be responsible for 100 or more different protein forms.

Functional analysis of the repertoire of expressed gene products will require efficient and rapid methods for discovery of protein-protein interactions. Integration of cell function depends on such interactions. Even when the complete repertoire of expressed gene products in humans becomes known in the near future, functional analysis of these gene products will still require identification and analysis of protein-protein interactions. Understanding these interactions will not only provide important information about normal development and physiology but will allow us to design rational therapies for human diseases. Specific protein-protein interactions are essential to cell function, and disruption of these interactions by mutation, pathogens or toxins, causes human disease. However, we are far from identifying and cataloguing the large number of these important interactions so that efficient and rapid methods to identify protein-protein interactions are among the important tools needed for efficient exploitation of the fruits of the human genome project(s). Peptide expression libraries are potentially useful for rapid screening of protein partners and identification and analysis of protein binding domains. Peptide display libraries, in which short, random peptide sequences are expressed at the surface of a bacteriophage, have been used extensively to identify peptide ligands for specific proteins such as signaling molecules, receptors and antibodies (Guarente, L., 1993, Proc. Natl. Acad. Sci. USA. 90: 1639-1641; Sparks, AB et al., 1998, Meth. Mol. Biol. 84:87-103; Kay, BK, 1995, “Mapping protein-protein interactions with biologically expressed random peptide libraries”. Persp. Drug Discov. Des. 2:251-268; and U.S. Pat. Nos. 5,837,500 and 5,403,484, all of which references are incorporated by reference in their entirety). In general, phage display is a powerful technique for identifying peptides or proteins that have sought-after binding properties. A peptide or protein is displayed on the surface of a bacteriophage as a fusion to a protein that is normally found in the phage particle. The earliest phage vectors for surface display were filamentous phage prepared by Smith and coworkers (Smith, GP et al., 1993, Meth. Enzymol. 217, 228-257). These investigators developed simple procedures for selecting phage displaying peptides or proteins that bind to pre-determined targets. Such phage can be selected readily from large libraries of variants. In this approach both the peptide or protein and its coding sequence are selected at the same time because the displayed peptide or protein responsible for binding is encoded in the genome of the bound phage. Phage display has been used to identify peptides that bind to receptors, substrates or inhibitors of enzymes, epitopes, improved antibodies, altered enzymes, and cDNA clones (O'Neil, K T et al., 1995, Current Opinion in Structural Biology, 5:443-449).

In one well-developed system, combinatorial peptides encoded by degenerate oligonucleotides are expressed as fusions with the N-terminus of the major or minor capsid proteins of M13 phage. Libraries with a diversity of 10⁸ to 10¹⁰ have been rapidly screened for a wide variety of interactions (Smith et al., 1997, Chem. Rev. 97:391-410). This serves as a powerful approach to analyze the constraints imposed on interactions and their affinity by changes in amino acid sequence (e.g., Chan et al., 1998, Meth. Mol. Biol. 84:75-86; Pierce et al., 1998, J. Biol. Chem. 273:23448-23453). The power of expression libraries as targets for identification of protein partners has been limited by the lack of a suitable host phage for efficient expression of cDNAs. Sporadic attempts have been made to screen λgt11 cDNA expression libraries for interacting partners (see Guarante, supra), but expression of target proteins in the bacterial host is inefficient and their availability following transfer to a suitable medium is compromised.

The yeast two-hybrid system is at present the only other system in which a “bait” protein may be screened against a cDNA library for potential interacting partners. The development of the present screening approach, while not replacing the two-hybrid system, represents an additional set of tools in our arsenal of methods in that it extends the potential and increases our capacity to screen many targets simultaneously.

The utility of the yeast two hybrid system has recently been extended to screen for multiple interactions by preparing a library of “baits” in one yeast strain and a library of potential interacting partners in a second. Mating of these strains can, in theory, generate all possible combinations of baits and partners and should be suitable to begin some bookkeeping (Kolonin et al., 1998, In: Current Protocols in Molecular Biology, Unit 20.1., and Current Protocols in Protein Science, Unit 19.1, John Wiley and Sons, Inc., New York, N.Y.). However, this system suffers from at least one weakness: the spurious activation or repression of transcription that occurs because, in the nucleus, 10 selection for interactors arises from the interaction of a known “bait” protein (fused to the DNA binding domain of the Gal4 promoter) with an unknown protein partner (fused to the activation domain) (Fields et al., 1989, Nature 340:245-247; Chien et al., 1991, Proc. Natl. Acad. Sci. USA. 88,9578-9582). This problem has been addressed with a newer two hybrid system based on activation of Ras by the human GDP-GTP exchange factor hSos (Aronheim et al., 1997, Mol. Cell Biol. 17:3094-3102). Activation can only occur when Ras is localized to the plasma membrane. Thus protein “baits” are fused to hSos and the cDNA library containing the putative partner is fused to a membrane localization signal. Interaction of hSos with a partner rescues the cdc25-2 phenotype. The general applicability of this system will have to await more extensive experience.

S. Michnick's group has described protein fragment complementation assays to detect 2Q biomolecular interactions in vitro or in vivo (PCT Publication WO9834120A1; ), Pelletier, J N et al., Nat Biotechnol 17(7):683-90 (1999); Remy, I et al., Proc Natl Acad Sci USA 96(10):5394-9 (1999).

Using murine dihydrofolate reductase (mDHFR) as an example, the method utilizes fusion peptides consisting of N and C-terminal fragments of murine DHFR fused to GCN4 leucine zipper sequences were coexpressed in E. coli grown in minimal medium, where the endogenous mDHFR activity was inhibited with trimethoprim. Coexpression of the complementary fusion products restored colony formation. Pelletier et al., supra, described a rapid, efficient in vivo library-versus-library screening strategy for identifying optimally interacting pairs of heterodimerizing polypeptides. Two leucine zipper libraries, semi-randomized at the positions adjacent to the hydrophobic core, were genetically fused to either one of two designed fragments of mDHFR), and cotransformed into E. coli. Interaction between the library polypeptides reconstituted enzymatic activity of mDHFR, allowing bacterial growth. Use of more weakly associating mDHFR fragments, increased the stringency of selection. Competitive growth allowed small differences among the pairs to be amplified, and different sequence positions were enriched at different rates. These selection processes were applied to a library-versus-library sample of 2.0×10⁶ combinations and selected a novel leucine zipper pair that may be appropriate for use in further in vivo heterodimerization strategies.

Sche, P. P. et al., Chem. Biol. 6:707-7166 (1999) disclosed a procedure of direct cloning of cellular proteins based on their affinity for natural products. See, also, C&EN, Oct 4, 1999, pp 33-34. This “display cloning” approach involves cloning of proteins displayed on the surface of a phage particle. The authors exemplified isolating of full length gene clone of FKBP-12 from a human brain cDNA library using biotinylated FK506 probe molecule. FKB12 was the dominant library member after affinity selection and was the only sequence identified after 2 rounds of selection. This method is said to allow amplification and repeated selection of putative sequences, leading to unambiguous target identification. This process eliminates the subsequent cloning step needed with affinity methods preformed on tissue homogenates of cell lysates.

Co-immunoprecipitation has been, and remains, an important technique for uncovering and verifying interacting systems of proteins. In some of the most important breakthroughs in unraveling the machinery behind specific cell function, immunoprecipitates formed by antibodies specific for a single component have been used to isolate complexes. The protein components of the complexes are then separated by polyacrylamide gel electrophoresis in the presence of sodium dodecyl sulfate (SDS PAGE) and the individual proteins identified by amino acid sequencing or tests with other available antibodies. Additionally, interactions initially identified using the yeast two-hybrid system (or other means), have been verified, and the antibody-based analysis of their physiological or developmental roles has been extended. The present invention exploits a similar strategy by preparing anti-peptide antibodies directed against putative partners that were identified in the T7 screen to verify and further analyze the molecular interactions.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

List of Abbreviations

The following are some of the non-standard abbreviations used herein:

gDP: genetic display package, such as a phage, that includes in its genome DNA encoding a heterologous peptide that is to be displayed on the surface of the package (e.g., phage)

OSP: outer surface protein (e.g., of a bacteriophage) that is to serve as a fusion partner for a PBD to be displayed on the phage; gene encoding OSP is designated osp.

PBD: potential binding domain of a protein (plural is “PBDs”); the “gene” encoding the PBD is in lower case italics (pbd); a fusion with an OSP is designated OSP-PBD

ΦDL: phage display library, which consists of phages expressing the library of PBDs as peptide sequences on their outer surface in the form of fusion proteins with a phage outer surface protein (“OSP”) and bind directly to a target epitope, preferably a peptide, permitting their isolation in batch.

General Discussion of Protein Domains

Most larger proteins fold into distinguishable structures called domains (Rossman, M et al., Ann Rev Biochem, 1981,50:497-532. A protein domain has been defined various ways: (a) in terms of 3D atomic coordinates, (b) as isolatable, stable fragment of a larger protein, and (c) based on protein sequence homology. This diversity of definitions relates to concepts of domains in predicting the boundaries of stable fragments and the relationship of domains to protein folding, function, stability and evolution. Herein, definitions of “domain” which emphasize retention of the overall structure, even in the face of perturbing forces such as elevated temperatures or chaotropic agents, are favored, though atomic coordinates and protein sequence homology are also considered. When a domain is primarily responsible for the protein's ability to specifically bind a target molecule, it is referred to herein as a “binding domain” (BD). One stage of this invention engineers the presence of a stable BD (denoted as a PBD; see above, on the surface of a gDP. For further description of domains, see, Janin, J et al., “Domains in Proteins: Definitions, Location, and Structural Principles”, Meth. Enzymol. (1985), 115(28):420-430; Rose, G D, “Automatic Recognition of Domains in Globular Proteins”, Meth. Enzymol. (1985), 115(29): 430-440; Rashin, A, Biochemistry (1984), 23:5518; Vita, C et al., Biochemistry (1984), 23:5512-5519.

Traditionally, partial proteolysis and protein sequence analysis was commonly used to isolate and identify stable domains. (See, for example, Vita et al., supra, Poteete, A R, J Mol Biol (1983), 171:401-5 418; Scott, M J et al. J Biol Chem (1987), 262:5899-5907. If the only structural information available is the amino acid sequence of the candidate OSP, this information can be used to predict turns and loops with high probability (Chou, P Y & Fasman, G D, “Prediction of protein conformation” Biochemistry (1974), 13:222-245; Chou, P Y & Fasman, G D, “Prediction of the secondary structure of proteins from their amino acid sequence”, Adv Enzymol (1978), 47:45-148; Chou, P Y & Fasman, G D, “Empirical predictions of protein conformation” Annu Rev Biochem (1978), 47:251-276.

Screening Method for Protein-Protein Interactions

The present inventors set out to perfect a methodology for screening protein-protein interactions that is rapid, easy and generally applicable to a wide array of such interactions. The present method permits one to catalogue protein-protein interactions rapidly and is amenable to full automation for large scale screening. By developing a novel adaptation and combination of certain existing technologies, the present inventors have created a high throughput screening methodology that can identify the particular amino acids or domains or epitopes that are of primary importance in the binding interactions between two protein partners. This permits (a) the recognition of developmentally and physiologically significant protein binding partners, (b) the rapid identification of the residues to and by which they bind, and (c) identification of protein-protein interactions that require, or occur under, specific environmental conditions (such as temperature, presence or absence of calcium, just to name a few).

The present methods have advantages over the prior art methods for discovery of protein partners that are labor intensive and time consuming and thereby constrain our ability, for example, to correlate loss of cell function with loss of specific protein-protein interactions. The methods of this invention are rapid, simple to use, and potentially automatable.

In a preferred embodiment, this invention entails simultaneous synthesis of numerous individual peptides of known sequence on a solid support array, such as on “Multipins” that are arrayed in a manner complementary to the wells of standard 96-well microplates. This is preferably done using the Multipin™ Peptide Synthesis Kit from Chiron or by similar methods such as those described in U.S. Pat. Nos. 5,266,684, 5,010,175, 5,182,366, 5,194,392 and 4,833,092. Other references that describe relevant methods for the synthesis and use of such peptide arrays are given below.

An array is preferably designed to contain sequentially overlapping short peptides are a part of a contiguous sequence of a protein (or protein domain) of interest. These peptides are targets for the binding of (or by) a potential binding domain (“PBD”) that is subjected to the screening and identification method of the invention; binding is preferably assessed using a modified enzyme-linked immunosorbent assay (ELISA), although other immunoassays and analytical techniques can be substituted. This method facilitates rapid identification of those amino acids (in the arrayed target peptides) that participate directly in, or are otherwise important for, the interaction between two proteins: the protein from which the target peptides are derived and the PBD of its binding partner.

The proteins being tested for the presence of a PBD by binding to the arrayed peptides are displayed on a “Genetic Display Package” (“gDP”) such as bacteriophages in the form of a phage display library (“ΦDL”), preferably a T7 ΦDL that comprises phage vectors that include in their genetic material a member of a cDNA library being sampled. The peptide targets are immobilized to a solid phase device, for example in 96 pin/well arrays, which displays them to the PBDs. This method has the potential to identify large numbers of interactions and to readily determine the amino acid domains, whether linear or conformational, through which the interactions occur.

The library of cDNA being displayed as PBDs is derived from a “biological source” which may be tissue, organ, cell population, cell line or other such source from which mRNA can be obtained. This approach permits sampling of the biological source at a specific developmental stage or in a particular physiological or pathological state. The gDPs, preferably phage particles, more preferably T7 phage. These phages express the library of PBDs as peptide sequences on their outer surface in the form of fusion proteins with a phage outer surface protein (“OSP”) and bind directly to a target epitope, preferably a peptide, permitting their isolation in batch.

The immobilized overlapping synthetic target peptides that represent specific sequences in the target protein of interest are used to sort the phage displaying surface PBDs into binding and nonbinding populations. The presence of bound phage particles indicates display of a peptide that interacts with the specific target amino acid residues in that well-residues that are a part of a predetermined domain or segment of interest of the target protein. Multiple rounds of selection can be carried out, comprising the steps of binding the phage to the target peptides, elution of bound phage, another round of growing the phage on appropriate bacterial hosts, and using the phage progeny to repeat the above steps.

The Examples below set forth the screening system and present in more detail the experimental systems uses to develop and test the methods of this invention.

The present methods exploit two relatively recent developments in the art: (1) the T7 phage expression system, and(2) a semi-automated (and potentially fully automatable) system in which peptides are synthesized while covalently attached to a 96 Pin support (readily expandable to 384 pins or greater). The present inventors have optimized, integrated and expanded the utility of these two technologies in a novel way. It is important to note that the present methods are not limited to PBDs that bind peptide epitopes, because other structures such as sugars and nucleic acids, if appropriately arrayed, can serve as targets as well.

Specifically, the present invention provides a screening method for identifying, in a library of potential binding domains (PBDs) from a biological source, a polypeptide binding domain or domains that bind to a target epitope or family of target epitopes, the method comprising:

(a) providing a cDNA library from the source that encodes the library of PBDs as a T7 phage display library (ΦDL) wherein the PBDs are displayed on the outer surface of the T7 phages as fusion proteins with an outer surface protein (OSP) of the T7 phages;

(b) contacting the ΦDL with a bindable array of target epitopes or families of epitopes under conditions where any of the PBDs binds to their target epitopes;

(c) removing unbound T7 phages from the array of target epitopes, so that phages remaining bound are a first sublibrary enriched for PBD-displaying phages;

(d) eluting bound T7 phage from the array of target epitopes ; and

(e) determining the DNA sequence encoding the PBDs from the first sublibrary of eluted T7 phage, thereby identifying the PBDs displayed on the eluted phage by their predicted amino acid sequence.

In the foregoing method, preferably at least one of (i) the PBDs of step (a), or (ii) the target epitope or family of step (b) are predetermined. More preferably, the target epitope or family of epitopes are predetermined.

After eluting step (d) and before the determining step (e), the invention preferably includes the step of:

(f) subjecting the eluted phage to at least one additional round of contacting and removing of steps (b) and (c) to further enrich phage displaying the PBDs that bind to set predetermined target epitope or epitopes, thereby obtaining a second sublibrary and subsequent sublibraries. Step (f) may be repeated more than once prior to the determining step (e), after each repeat obtaining a new subsequent sublibrary.

In the foregoing method, the outer surface protein is preferably capsid protein encoded by gene 10A or 10B of phage T7, more preferably, the 10B-encoded protein.

In the above method, in the display library, the PBDs are may be expressed in a copy number of about 5-10 PBDs per phage particle, or alternatively, at a high copy number of 415 PBDs per page particle. In other embodiments, the PBDs are expressed in an intermediate copy number of about 100 to about 150 PBDs per page particle.

In the present methods, the determining step (e) is preferably performed by plating the eluted phage on a lawn of E. coli, permitting them to multiply and form plaques, and sequencing the DNA of the phages of any given plaque to obtain the sequence of the cDNA insert that encodes the PBD.

The target epitopes indicated above are preferably peptide epitopes and the family preferably comprises peptides or polypeptides corresponding to (i) a protein fragment, (ii) a protein domain or 25 (iii) a complete protein. The family preferably comprises a progressive series of overlapping peptides of about 10 to 15 amino acids, each of which peptides lacks n amino-terminal amino acid residues of its predecessor peptide in the series and has at least n additional amino acids added to its carboxy-terminus, wherein n is an integer between 1 and 5, and wherein the series of overlapping peptides corresponds to (i) a region of the protein of up to about 100 amino acids, or (ii) the complete protein.

The target peptides are preferably synthesized in parallel on polyethylene pins mounted on blocks which are compatible with standard microplate arrays of 96 wells or multiples thereof. The target peptides are preferably covalently attached to the pins so that the, after the eluting of the bound phages, the blocks may be reused for one or more additional screening assays. The target peptides may be in a cleavable form, allowing recovery of the peptides.

In another embodiment of the above method, the cDNA library is produced from mRNA molecules of the biological source by random priming wherein each cDNA molecule reverse transcribed from the mRNA molecules is between about 50-5000 bp in length, preferably 50-1000 bp, more preferably 50-500, more preferably 100-200 bp. The cDNA molecules are preferably gel purified and directionally cloned into the T7 phage DNA resulting in fused DNA which is packaged into phage in vitro.

The present invention is further directed to a method to determine the representation of expressed sequences in a PBD display sublibrary, when the PBDs are from a known protein and specific antibodies for epitopes of the known protein are available,

(i) providing a collection of antibodies specific for the epitopes of the known protein which antibodies are immobilized to a solid support, preferably magnetic beads;

(ii) carrying out the method of claim 5 or 6 up to an eluting step wherein the first sublibrary, the second sublibrary or a subsequent sublibrary is obtained;

(iii) contacting the sublibrary obtained in step (ii) with the antibodies of step (i) and permitting the antibodies to bind to the epitopes of the displayed PBDs

(iv) evaluating the results of the binding, thereby determining the representation of the expressed sequences in the sublibrary.

In addition to the antibody binding steps, this method may include the step of obtaining multiple separate phage clones from the sublibrary, separately isolating the DNA therefrom, and sequencing the cDNA insert of each clone that encodes the PBD of that clone.

Preferred biological sources for the above methods include developing chick neural retina, cultured neonatal rat Schwann cells, and myelinating sciatic nerves of 15-day old rat. When using Schwann cells or sciatic nerves, preferred target epitopes are peptides of a peripheral myelin protein selected from the group of proteins consisting of PMP22, P0 (e.g., a cytoplasmic domain of P0), connexin 32 and EGR2.

In another embodiment, the (DDL displays PBDs of a protein selected from the group consisting of β-catenin, PTP1B, p120ctn and She; and the target epitopes are peptides of N-cadherin. In yet another embodiment, the ΦDL displays PBDs of synaptotagmin SytI and the target epitopes are peptides of synaptotagmin Syt IV; or the ΦDL displays PBDs of SytIV and the target epitopes are peptides of Syt I. In another embodiment, ΦDL displays PBDs of SytI or Syt IV and the target epitopes are peptides of syntaxin; or the ΦDL displays PBDs of syntaxin and the target epitopes are peptides of Syt I or Syt IV.

A method of identifying peptides participating in protein-protein interactions by screening a first peptide display library for members that interact with a second peptide display library, the method comprising

(a) providing a first cDNA library from a biological source that encodes PBDs as a first T7 ΦDL wherein the PBDs are displayed on the outer surface of the T7 phages as fusion proteins with an outer surface protein of the T7 phages, which first display library is immobilized to a solid support, and the PBDs are available for binding to a peptide or a protein domain for which they have binding specificity;

(b) providing the second library which is a combinatorial library of peptides displayed on genetic display packages (gDPs) other than T7 (preferably also phage, most preferably M13) that are available for binding to the immobilized members of the first library;

(c) contacting the members of the immobilized T7 first library with members of the second library;

(d) removing unbound particles of both of the libraries so that second library particles remaining bound are enriched for those displaying peptides that bind to the PBDs displayed on the T7 phages,

(e) eluting the bound particles

(f) selectively growing the T7 phages and the gDPs under conditions wherein either the T7 phages or the gDPs have a growth advantage to obtain enriched populations of the T7 phages expressing the first library and the gDPs expressing the second library;

(g) separately amplifying the DNA of the second library particles and the immobilized first library phages to which the second library particles had been bound, and sequencing amplified DNA libraries, thereby determining the predicted amino acid sequences of

(i) the PBDs normally expressed in the biological source that participate in the protein-protein interactions with the second library peptides, and

(ii) the peptides that are part of, or that mimic, endogenous proteins that normally interact with the first library PBDs

thereby identifying the peptides participating in the protein-protein interactions

In this method, immobilization is preferably achieved using an antibody specific for an outer surface structure of the T7 phage, preferably a tail fiber.

In the foregoing method, the gDP is preferably M1 3 and the second library is an M1 3 random combinatorial peptide library. Preferably members of the second library have from about 4 to about 30 amino acids with a complexity of expressed peptides of between about 10⁷ and about 10¹⁵.

### BRIEF DESCRIPTION OF THE DRAWINGS

FIG. 1 illustrates in schematic form the host and vector elements available for control of T7 RNA polymerase levels and the subsequent transcription of a target gene in a pET vector.

FIG. 2A, B, C illustrates the integration of T7 capsid expression and synthetic peptide “panning” into a screening procedure.

FIG. 2A describes proteins expressed as fusions with Glutathione-S-Transferase in E. coli and immobilized on glutathione magnetic beads.

FIG. 2B shows pins bearing target sequences recognized by a binding domain displayed on T7 bind many phage encoding overlapping sets of cDNA sequences.

FIG. 2C illustrates how, as one moves along the Pin array representing a protein target, there are increases and decreases in the number of plaques formed by the eluted phage consistent with the distribution of binding domains

FIGS. 3 and 4 are SDS-PAGE electropherograms (autoradiographs) illustrating the oligomerization properties of Syt IV with Syt I. FIG. 3 shows that, in the presence of calcium, GST alone or the C2A domain of Syt IV essentially does not bind with Syt I or Syt IV.

FIG. 4 shows that, in the presence of calcium, both immobilized recombinant Syt I and Syt IV C2B domains interact with in vitro translated Syt I and Syt IV.

FIG. 5 shows a diagrammatic representation of peptide-protein binding and ELISA assay.

FIG. 6 shows a diagrammatic representation of spacer insertion and negative selection system.

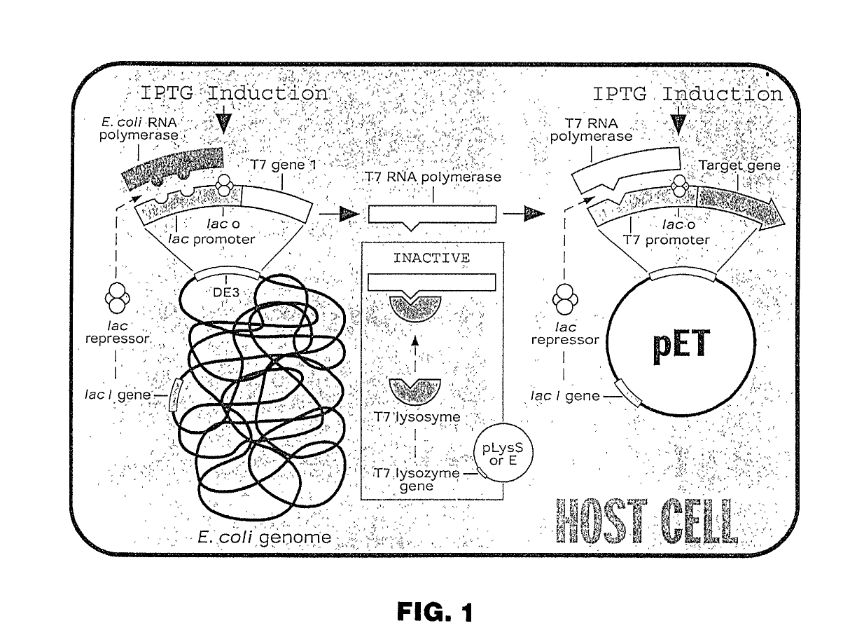

### DESCRIPTION OF THE PREFERRED EMBODIMENTS

General methods and information for the methods and materials described herein may be found in references well-known to those skilled in the art, for example, Atherton and Sheppard, 1989, Solid Phase Peptide Synthesis,—A Practical Approach, IRL Press, Oxford, U.K., 1989; two books by Bodansky, M. and Bodansky, A.: The Principles of Peptide Synthesis and The Practice of Peptide Synthesis, Springer-Verlag, London, 1984; Greenstein J P and Winitz, M., 1961, Chemistry of the Amino Acids, Wiley, New York, 196 1; Gross et al., eds. The Peptides—Analysis, Synthesis and Biology, volumes 1-9, Academic Press, New York, 1979-1989; Porter, Ret al., eds., 1986, Synthetic Peptides as Antigens, Ciba Found. Symp. 119 (especially pp. 130-149). Publications by H. M. Geysen and his colleagues describe the methods of overlapping peptide analysis, including solid phase peptide synthesis, peptide arrays, screening for peptide binding, recognition of peptide epitopes by antibodies, and the like. Preparation of target peptide libraries for the present invention employ such methods; many aspects are covered in: Bray, A M et al., 1990, Tetrahedron Lett. 31 :5811-5814; Bray, AM et al., 1991, Tetrahedron Lett. 32:61631-6166; Bray, A M et al., 199 1, J. Org. Chem. 56:6659-6666; Maeiji, N J et al., 199, Peptide Research 4:142-146; Maeiji, N J et al., 1992, J. Immunol. Meth. 146:83-90; Valerio R M etal., 1993, Int. J. Peptide Prot. Res. 42:1-9; Geysen 1990, Southeast Asian J. Trop. Med. Pub. Health, 12:523-533; Geysen et al., 1988, J. Mol. Recog. 1:320-341; Geysen et al., in Molecular Mimicry in Health and Diseases, 1988, Elsevier, Amsterdam; Geysen et al., 1987, J. Immunol. Meth. 102:259-274. All the foregoing references are incorporated by reference in their entirety.

The cloning and peptide technology initially used by the present inventors was based on a system of partially characterized protein interactions: the binding of effectors to the cytoplasmic domain of N-cadherin. Four known effector/adaptor molecules are known to bind to the cytoplasmic domain of N-cadherin: p120ctn, Shc, PTP1B, and β-catenin. The target sequences in N-cadherin for three of these proteins have been localized to regions of between 30 and 50 amino acids. Use of this model serves to demonstrate the efficacy of this invention, as well as permitting the refinement of target sequences for each of the interacting proteins.

The present method is also applied in a model system that is relevant to the field of toxicology—the Ca²+-dependent interaction of synaptotagmin with binding partners during neurotransmitter secretion Characterization of this interaction and the amino acids involved will serve future research on lead (Pb²⁺) toxicity which may be mediated in part by disruption of synaptotagmin binding.

This invention (a) optimizes the synthesis and cloning of the appropriate length cDNAs for capsid expression in T7, and (b) optimizes the length and overlap of synthetic peptides to pinpoint the binding region for clones expressing binding partners.

To test the efficacy of the system to discover an unknown interaction or interactions, the present inventors use the major structural proteins of peripheral nerve myelin as targets for novel interacting gene products. Peripheral myelin proteins have been extensively characterized and cloned, and many point mutations are known that cause severe demyelinating disease. However, the regulation of assembly and function of these proteins during myelination remains obscure, and effector/signaling molecules remain to be identified.

T7 Expression Library from Myelinating Rat Sciatic Nerve

The combination of T7 capsid expression and synthetic peptide “panning” (described below) leads to identification of novel “adaptor” or “effector” proteins as exemplified in myelinating Schwann cells.

A T7 expression library from myelinating rat sciatic nerve will be constructed in T7 phage.

Overlapping peptides representing the cytoplasmic domains of the four proteins P0, PMP22, Cx32 25 and EGR2 will serve as the targets. cDNA inserts from phage that interact with target peptides will be sequenced and compared to each other and to sequences in existing data banks. Those DNA sequences from phage having identical or overlapping inserts that bound to a specific target amino acid sequence will be examined by Northern blots for up-regulation during myelination.

Antibodies specific to the peptides will be prepared by conventional means and will be used to analyze the peptides' cellular location and in situ associations.

Sequences of potential interest for which suitably immunogenic regions have not been identified or for which additional sequence information is not present in existing data bases, will be 5 used for isolation of additional or full length sequences. Inverse PCR using existing libraries is a preferred method of generating additional sequence; alternatively, 5′ or 3′ RACE. This obviates the need for a library. Given that the original clones were generated from Schwann cell mRNA, it is possible, using the same mRNA preparation methods described herein, to amplify additional sequences. Although characterization of full length clones is desirable, it may not be a primary goal. However, it is preferred to obtain enough sequence for designing peptide to produce antibody probes for analyze the biology of the molecules discovered by the present methods.

General Aspects of the T7 Expression System

Studier and colleagues developed an improved phage display system using the well-characterized bacteriophage T7 (described below). This system is easy to use and has the capacity to display peptides up to about 50 amino acids in size in high copy number (415 per phage), and peptides or proteins up to about 1200 amino acids in low copy number (5 -10/phage) in the form of fusion products with the phage capsid protein. T7 is a well-characterized double-stranded DNA phage (Dunn, J J et al., 1983) J. Mol. Biol. 166, 477-535; Steven, AC et al., 1986) Electron Microscopy of Proteins 5:1-35). Phage assembly takes place inside E. coli bacterial cells, and mature phage are released by cell lysis. Unlike the filamentous phage systems described below, peptides or proteins displayed on the T7 surface do not require prior secretion through the cell membrane, a necessary step in filamentous phage assembly (Russel, M., 1991, Mol. Microbiol. 5:1607-1613). The relatively new “T7 Select™” expression system combines the power of phage expression with cDNA expression.

T7 is an attractive display vector because it is very easy to grow and replicates more rapidly than either bacteriophage λ or filamentous phage. This system has a number of advantages over an earlier system based on M13 phage. M13 phage must be secreted through the bacterial coat. In contrast, T7 is a lytic phage that grows rapidly on bacteria, forms plaques within 3 hrs at 37° C., and cultures lyse 1-2 hours after infection, decreasing the time needed to perform the multiple rounds of growth usually required for selection. The T7 phage particle is extremely robust and is stable to harsh conditions that inactivate other phage. This expands the variety of agents that can be used in bioaffinity-based selection procedures which require that the phage remain infective. T7 is an excellent general cloning vector. Purified DNA is easy to obtain in large amounts, a high-efficiency in vitro packaging system is available (Son, M et al., 1988, Virology 162, 38-46), and the phage genome DNA (39,937 bp) has been completely sequenced, making restriction or DNA sequence analysis of clones quite straightforward.

T7 structure and assembly

T7 is an icosahedral phage with a capsid shell composed of 415 copies of the T7 capsid protein (gene 10) arranged as 60 hexamers on the faces of the shell and 11 pentamers at the vertices (Steven, A C et al., 1986, Electron Microscopy of Proteins, 5:1-354). Attached at the remaining vertex is the head-tail connector (gene 8), a short conical tail (genes 11 and 12) and 6 tail fibers (gene 17). The phage assembly process is similar to that of other double-stranded DNA phages (Cerritelli, M E et al., 1996, J. Mol. Biol. 258:286-298). DNA is packaged into a procapsid shell made up of scaffolding protein (gene 9), capsid protein, the head-tail connector, and an internal protein structure (genes 13, 14, 15, and 16). The DNA is packaged from linear concatemers, and as the DNA enters the procapsid shell, the scaffolding protein is released causing a conformational change in the shell to form the mature particle. Tail and tail fibers attach at the head-tail connector vertex.

The T7Select™ Phage Display System uses the T7 capsid protein to display peptides or proteins on the surface of the phage. The capsid protein is normally made in two forms, “10A” (344 aa) and “10B” (397 aa). Form 10B is produced by a translational frameshift at amino acid (aa) 341 of 10A, and makes up about 10% of the capsid protein (Condron, B G et al., 1991, J Bacteriol. 173:6998-7003). Functional capsids can be composed entirely of either 10A or 10B, or of various ratios of the proteins. This finding provided the initial suggestion that the T7 capsid shell could accommodate variation, and that the region of the capsid protein unique to 10 B might be on the surface of the phage and could be exploited for phage display.

T7Select™ vectors

Two basic types of T7Select™ phage display vectors are available: the T7Select415 vector for high-copy number display of peptides, and the T7Select1 vectors for low-copy number display of peptides or larger proteins (see Table below).

Phage display vector features

| Vector | Use | Display # | Display Limit | Host |
|---|---|---|---|---|
| T7Select415-1 | peptides | 415 | 40-50 aa | BL21 |
| T7Select1-1 | peptides or | ≦1 | 900 aa | BLT5403 |
| | proteins | | | |
| T7Select1-2 | peptides or | ≦1 | 1200 aa | BLT5403 |
| | proteins | | | |

In all of the vectors, coding sequences for the peptides or proteins to be displayed are cloned within a series of multiple cloning sites following the codon for aa 348 of the 10B protein. The natural translational frameshift site within the capsid gene has been removed, so only a single form of capsid protein is made from these vectors.

Functional peptides up to 39 amino acids have been displayed from T7Select415™. Expression of the T7Select415™ capsid gene is controlled by the Owild-type strong phage promoter (Schmidt, T G et al., 1993, Protein Eng. 6:109-122) and translation initiation site (s10), and the capsid/peptide fusion protein is produced in large quantities during infection. T7Select415™ clones generally grow well on normal laboratory hosts such as E. coli BL21. The capsid shell is composed entirely of the capsid/peptide fusion protein so that 415 copies of peptide are displayed on the phage's surface. High copy number display is desirable wherever a strong signal is useful, such as in epitope mapping. It is also preferred for displaying peptides that bind weakly to their targets.

Functional proteins having as many as about 1000 amino acids have been displayed from T7Select1-1™ vectors. The T7Select1-2a,b,c series provides multiple cloning sites in all three reading frames and includes a blunt-end site (EcoRV). Peptides or proteins are displayed in low copy number (about 0.1-1 per phage) from these vectors, which makes them suitable for the selection of proteins that bind with high affinity to their targets. To obtain low-copy display, the promoter of the capsid gene was removed and the translation initiation site was altered. The capsid mRNA is still controlled by phage promoters located further upstream of the gene, but production of capsid protein is greatly reduced. T7Select1™ phages are grown on a complementing host (BLT5403) that provides large amounts of the 10A capsid protein from a plasmid clone. The 10A gene in the complementing plasmid and the capsid gene in the vectors are engineered to minimize any recombination between them.

Cloning in T7Select vectors

Cloning in T7Select™ vectors utilizes procedures similar to those for cloning in phage λ vectors. Vector arms are prepared and ligated with target inserts, the resulting DNA is incubated with an in vitro packaging extract, and the phage products are used to infect a suitable host. The multiple cloning sites in the T7 vectors are compatible with many existing vectors, including the pET vectors that are most suitable in T7 expression system for the present invention (described below).

The DNA inserts usually contain a limited region encoding variant amino acids. Obviously, the size of the library required to have a good chance of including all variants increases with the number of varied amino acids. For example, a complete heptapeptide library has 20⁷=1.28×10⁹ unique heptapeptides. The capacity to construct large libraries in any cloning system depends on the efficiency of cloning and packaging (phage) or transformation (plasmids). The vector arms and T7 packaging extracts in the T7Select™ System routinely produce >10⁸ recombinant plaques per μg of arms. This efficiency is 10- to 50-fold higher than observed with most cloning systems and is comparable to the optimal efficiency of plasmid systems. The high-efficiency T7 packaging extracts (2×10⁹ plaques per μg intact DNA) are made with a specially designed phage that reduces the non-recombinant cloning background to below 0.1%.

For verification of performance, one can use commercially available kits such as T7Select™ Cloning Kits from Novagen. These include a positive control target DNA, which encodes the 15 aa S•Tag™ peptide. S•Tag recombinants are easily detected with a rapid, chemiluminescent plaque lift assay using the T7Select™ Biopanning Kit.

A variety of biologically active peptides and proteins have been displayed from the T7Select™ vectors. Those displayed in high copy number (415 per phage) include: S•Tag (15 aa) from pancreatic ribonuclease A; HSV-Tag™ epitope (11 aa) from Herpes Simplex Virus glycoprotein D; Streptavidin-binding peptide (10 aa) (Schmidt et al., supra); RGD peptide (8 aa) from adenovirus penton protein (Bai, M et al., 1993, J. Virol. 67, 5198-5205); thrombin cleavage site (7 aa) from pET vectors and HSV•Tag +His•Tag™ sequences (39 aa). Peptides such as the foregoing are cloned on DNAs that end up adding from about 10-39 aa to the 10B capsid protein (measured from the last naturally occurring aa, 348,). In each case, the display of functional peptide is verified by an appropriate binding assay. The use of the thrombin cleavage site enabled the direct demonstration that all 415 copies of peptide appear to be on the surface of the phage and were susceptible to being clipped off by thrombin without reducing phage infectivity.

T7Select vector cloning regions are shown below:

(1) T7Select415-1b, T7Select1-1b [SEQ ID NO:1 and 2]

| | |
|---|---|
| aa348 aa363 | |
| ...MetLeuGlyAspProAsnSerSerSerValAspLysLeuAlaAlaAlaLeuGlu | (SEQ.ID NO:2) |
| ...ATGCTCGGGGATCCGAATTCGAGCTCCGTCGACAAGCTTGCGGCCGCACTCGAGTAACTAGTTAA | (SEQ.ID NO:1) |
| BamHI EcoRI SacI SalI HindIII NotI XhoI | |

(SEQ. ID NO:1 is the nucleotide and SEQ ID NO:2 is the amino acid sequence)

```
            aa348 aa368
...MetLeuGlyGlySerAspIleGluPheGluLeuArgArgGlnAlaCysGlyArgThrArgValThrSer
...ATGCTCGGTGGATCCGATATCGAATTCGAGCTCCGTCGACAAGCTTGCGGCCGCACTCGAGTAACTAGTTAA
                BamHI EcoRV EcoRI SacI SalI HindIII NotI XhoI
```

(SEQ. ID NO:3 is the nucleotide and SEQ ID NO:4 is the amino acid sequence)

```
    aa348 aa365
...MetLeuGlyAspProIleSerAsnSerSerSerValAspLysLeuAlaAlaAlaLeuGlu
...ATGCTCGGGGATCCGATATCGAATTCGAGCTCCGTCGACAAGCTTGCGGCCGCACTCGAGTAACTAGTTAA
                    BamHI EcoRV EcoRI SacI SalI HindIII NotI Xho I
```

(SEQ. ID NO:5 is the nucleotide and SEQ ID NO:6 is the amino acid sequence)

```
            aa348 aa366
...MetLeuGlyIleArgTyrArgIleArgAlaProSerThrSerLeuArgProHisSerSerAsn
...ATGCTCGGGATCCGATATCGAATTCGAGCTCCGTCGACAAGCTTGCGGCCGCACTCGAGTAACTAGTTAA
                    BamHI EcoRV EcoRI SacI SalI HindIII NotI XhoI
```

(SEQ. ID NO:7 is the nucleotide and SEQ ID NO:8 is the amino acid sequence)

Peptides or proteins that have been displayed in low copy number (0.1-1 per phage) include: E. coli β-galactosidase (“β-gal”)(1015 aa); T7 RNA polymerase (873 aa); scFv single-chain antibody (257 aa); T7 endonuclease (149 aa); S•Tag (15 aa); and HSV•Tag (11 aa). For each, display was verified by either a binding assay or an enzymatic assay. Phage-displayed T7 endonuclease appeared to have about the same enzymatic activity as purified T7 endonuclease (De Massy, B et al., 1987) J. Mol. Biol. 193:359-376). The activity of β-gal phage is easily detected using a standard enzymatic assay (but was found to be about 250-fold lower than the measured copy number of the β-gal, presumably because β-gal is enzymatically active only as a tetramer.

It is unlikely that all displayed enzymes will be active “phagezymes.” Activity will depend on (a) whether the enzyme can maintain activity as an N-terminal fusion and, (b) where the phage has been purified, whether the enzymatic activity survives the purification process. For example, phage displaying T7 RNA polymerase were recognized by polyclonal antibodies to the polymerase while enzymatic activity for the phage was not observed.

Panning Selection

A preferred method for selecting phage displaying the desired PBD is by panning, coupled with growth of the phage enriched at every round. This method can yield nearly 10⁶-fold enrichment after two rounds with phage displaying the S•Tag in high copy number or the HSV•Tag in low or high copy number. S•Tag phage yielded a nearly 10-fold enrichment after two rounds.

The method has allowed >10⁷-fold enrichment after four rounds when the displaying phage had been mixed with control phage in a ratio of 1:2×10⁷.

The stability of the T7 phage particle enables the use of a variety of elution conditions during panning. The phage maintains infectivity following treatment with 1% SDS, SM NaCl, up to 4M urea, 2M guanidine-HCl, 10 mM EDTA, reducing conditions (up to 100 mM DTT), and alkaline conditions (up to pH 10). T7 phage are not stable to pH below about 4, which was a condition often used in panning filamentous phage (and may be exploited in the present invention for screening binding interactions between two sets of PBDs where neither is known, as is discussed below). For success both binding and elution conditions must preserve phage infectivity. Because of the wide range of conditions available for T7Select™, panning should permit enrichment of a wider variety of targets. The commercially available T7Select Biopanning Kit provides materials for testing a panning procedure using phage displaying the S-Tag peptide.

Methods based on “specific” elution are also included; these have the advantage of eliminating or reducing background. For example the displayed target protein may be immobilized to a solid matrix through a noncovalent linkage. For example, the displayed target protein may be in the form of:

(a) GST fusion protein which binds to a glutathione group on the matrix; or

(b) a His-tagged fusion protein which binds to Ni atoms on the matrix

The phage displaying the target fusion protein can be eluted using very specific conditions (e.g. excess glutathione+EDTA in (a) or an imidizole group (b)) leaving behind those bound phage particles which had bound nonspecifically to the matrix.

Large proteins cannot be cloned in the high copy number display vector (T7Select415TM).

Peptides up to at least 50 amino acids are expected to work because a displayed peptide of this size will create a capsid protein which is about the same length as wild-type T7 10B protein. The capacity of this vector system is sufficient for displaying structurally constrained peptides and peptides whose biological activity requires longer stretches of amino acids.

T7Select415™ phage are normally grown on the E. coli host BL21, where the fusion protein is the only source of capsid protein. Any growth inhibition that occurs may be relieved by growing the phage on BLT5403 cells which contains a plasmid that provides large amounts of 10A capsid protein. The capsid shell of phage produced in this manner will be composed of a mixture of intact 10A protein and the 10B fused with the protein/peptide library members.

The largest protein known to have been displayed on low copy display vectors is 1015 amino acids in length. The primary limitation on size is the DNA cloning capacity of the vector (e.g., 3.6 kbp, 1200 aa for T7Select11-™0 and 2.7kbp, 900 aa for T7Select1-2™ vectors). Phage displaying proteins of >600 amino acids may grow poorly, consistent with observations of the behavior of phage displaying a variety of proteins.

Phage that grow poorly must be grown on a complementing host (such as BLT5403) that provides the 10A protein (encoded by a plasmid) under control of a T7 promoter. Growth inhibition can be relieved by growing the phage on BLT5 615 cells, where plasmid expression of gene 10A is controlled by a different promoter (the lacUV5 promoter).

The absolute maximum copy number that is displayable on T7Select41 5™ phage grown on BL21 is limited to 415, the number of capsid proteins in the T7 shell. The maximal display number from low copy vectors is not similarly fixed, but also depends on several factors: (a) the ratio of expression of the capsid fusion protein from the vector and the 1 OA protein from the complementing 5 host (e.g., BLT5403 or BLT5615); and (b) the efficiency of assembly of the fusion protein into the capsid shell. Examples of actual copy numbers displayed per phage (as measured by Western blots) ranged from 0.5 down to 0.1.

A population of cDNAs from a tissue source, a cell population, a cell line or any other source can be cloned into the T7 phage and the products of this cDNA displayed on the phage 10 surface. Such displayed proteins or peptides are screened for the presence of peptide binding partners—preferably using known proteins or fragments as targets. Therefore the expressed polypeptides in the phage population represent the range of mRNAs that were expressed in the source tissue or cell; these polypeptides are of sufficient length (from ˜50 to over 1000 amino acids) to represent actual binding domains. Examples of know binding domains are SH2 (1000 amino acids) and SH3 (˜60 amino acids) (Src homology domains) and PDZ (˜80 amino acids).

The present inventors have conceived that the combination of the two systems, the T7 phage display system together with immobilized, arrayed protein/peptide targets, is an effective novel tool for discovering new protein-protein interactions.

Screening “Double Unknowns:” Combining the T7 cDNA Protein Display with a Random Peptide Display Expressed on the Surface of a Different “Genetic Display Package” (gDP)

Using the methods and tools described above, a cDNA library from a tissue, cells, an organ or an organism, is expressed in T7 such that the encoded proteins or peptide products, PBDs, of that library are displayed at the phage surface where they are free to interact with target protein or peptides with which they are capable of binding when those partners are presented or displayed in any of a number of different formats.

The approaches described above are directed at screening such T7 cDNA display libraries against synthetic peptides representing overlapping segments of known proteins of interest. This technology will identify cDNAs encoding PBDs which interact with the target peptides that preferably are chosen to represent physiologically and/or developmentally important signaling intermediates.

In addition to the foregoing, the present approach can be instituted as a general screen for protein-protein interactions in the case that neither specific binding partner is known. This method employs two gDP's, preferably different bacteriophages, that can be distinguished physically and separated one from the other. Two potentially interacting protein partners from two sources, e.g., different tissues, are displayed as separate cDNA display libraries, each library displayed in a different gDP. Different phages and even non-phage gDP's will be described below.

In one embodiment of this approach, a first display library, preferably a T7 cDNA display library, is immobilized through the phage tail fibers in a convenient format, e.g., a 96 well-format pin apparatus or other equivalent apparatus. One way to accomplish this is by first by immobilizing to the surface of the pins an antibody, such as a monoclonal antibody, specific for part of the phage that, when bound, will not interfere in the phage's peptide display and subsequent protein-protein interaction. A good candidate for this immobilization in T7 is the phage tail fiber protein. The anti-tail fiber antibody-coated pins are incubated with the T7 phage at an -appropriate dilution resulting in immobilization of T7 phage particles (the first interacting library).

The pin apparatus with the immobilized T7 display library is then screened against an combinatorial peptide library that is displayed on the surface of a different gDP, for example, M13 phage.

In another embodiment, the T7-PBD immobilized on pins are dipped into a batch fluid (rather than individual wells) containing a random peptide library (e.g., M13-peptide library. The pins, which have now bound complexes of T7-PBD-peptide-M13, are lifted out. The phage display complexes are eluted under conditions which may be harsh to maximize efficiency of elution. The two phage-displayed protein populations must be cloned and separated; this can be accomplished in several possible ways.

Selection of the M13 phage is performed by growth on a selective host that lacks T7 polymerase (e.g., Novagen pET system). The T7 phages are mutants in the polymerase to begin with. In the absence of the polymerase, only M13 phage will grow (not as lytic bursts but rather extruded through the bacterial membrane/cell wall.

To select the T7 “partner,” phage are grown in a host that provides T7 RNA polymerase.

After screening, the population can be passaged through T7 polymerase-negative hosts.

In summary, the population of phages obtained from the pins are grown on T7⁺M13⁻ hosts (where ⁺ indicates permissive and ⁻ indicates restrictive) vs. T7-M13⁺ hosts.

Screening on Mammalian Cells

The T7-PBDs are used in a screen employing mammalian cells that are maintained in suspension or are adherent, allowing identification of unknown ligands/receptors for these PBDs.

A bulk random T7 library is mixed with a bulk population of cells. T7 will be bound to those cells with cognate molecules for the PBD. To remove unbound phages, the cells are washed, e.g., by centrifugation in the case of suspended cells. The cell mixture with bound phages is lysed and plated on E. coli. Phage plaques are isolated and the inserts sequenced. Again M13 growth does not result in plaque formation because the M13 DNA is in the form of a plasmid. M13 normally does not grow as a virus unless a helper virus is provided. So selection is effected by picking and growing colonies expressing M13 DNA.

In another embodiment, the cells, e.g., COS cells, are engineered to overexpress a particular gene or a cDNA library against which one wishes to screen the phage display library.

Bacteriophages as gDPs

Bacteriophages are preferred gDPs because there is little or no enzymatic activity associated with intact mature phage and because their genes are inactive outside a bacterial host, rendering the mature phage particles metabolically inert. The filamentous phages (e.g., M13) are of particular interest. Other filamentous phage that may be used in the present methods include f1, fd, If1, Ike, Xf, Pf1, and Pf3.

For a given bacteriophage, the preferred outer surface protein (OSP) is usually one that is present on the phage surface in the largest number of copies, as this allows the greatest flexibility in varying the ratio of OSP:PBD and also gives the highest likelihood of obtaining satisfactory affinity separation. A protein present at low abundance is usually one that performs an essential function in the phage life cycle so that its alteration by addition or insertion of a peptide is more likely reduce phage viability. An OSP such as M13 gIII protein is a preferred choice for display of a PBD.

The user must choose a site in the candidate OSP gene for inserting a PBD gene fragment.

The coats of most phage are highly ordered. Filamentous phage have a helical lattice whereas isometric phage have an icosahedral lattice. Each copy of each major coat protein sits on a lattice point and has defined interactions with its neighbors. Proteins that make some, but not all, of the normal lattice contacts are likely to destabilize the virion. Thus in phage (unlike bacteria and spores as gDPs, see below), it is important to retain in an engineered OSP-PBD fusion protein those residues of the parental OSP that interact with other proteins in the virion. For M13 gVIII, it is preferred to retain the entire mature protein, whereas for M13 gIII it may suffice to retain the last 100 residues (or even fewer). Such a truncated gIII protein would be expressed along with the complete gIII protein, as gIII protein is required for phage infectivity. Il'ichev, AA et al. Dokl Akad Nauk SSSR, 1989, 307(481-483) reported viable phage having alterations in gene VIII but did not report on any binding properties of the modified phage nor did they insert a PBD or nor suggest that one be inserted.

Filamentous Phage

A filamentous phage, particularly M13, is preferred because:

(1) the external 3D structure is known;

(2) the processing of the coat protein is well understood;

(3) the genome is expandable;

(4) the genome is small;

(5) the genomic sequence is known;

(6) the virion is physically resistant to shear, heat, cold, urea, guanidinium HCl, low pH, and high salt;

(7) the phage is used as a sequencing vector so that sequencing is especially easy;

(8) antibiotic-resistance genes have been cloned into the genome with predictable results (Hines, J C et al., Gene, 1980, 11:207-218);

(9) It is easily cultured and stored (Fritz, H-J, IN: “DNA Cloning, D M Glover, ed., IRL Press, Oxford, UK, 1985), with no unusual or expensive media requirements for the infected cells,

(10) It has a large burst size, each infected cell yielding 100 to 1000 progeny particles after infection; and

(11) It is easily harvested and concentrated (Salivar, W O et al., 1964, Virology 24:359-371; Fritz, supra).

In addition to M13, other filamentous phage that may be used in the present methods include f1, fd, If1, Ike, Xf, Pf1 and Pf3. M13 and f1 are so closely related that properties of each is applicable to the other (Rasched, I., et al., 1986, Microbiol Rev 50:401-427). The genetic structure of M13, including the nucleic acid sequence (Schaller, H et al., in The Single-Stranded DNA Phages, Denhardt, DT et al., eds., Cold Spring Harbor Laboratory Press, 1978, p 139-163), the identity and function of the 10 genes, the order of transcription and the location of the promoters, is well known as is the physical structure of the virion (See Rasched et al., supra, for review). Because the genome is small (6423 bp), cassette mutagenesis is practical on RF M13 (Ausubel, F M et al., eds, Current Protocols in Molecular Biology, Greene Publishing Associates and Wiley-Interscience, Publishers: John Wiley & Sons, New York, 1987, as is single-stranded oligonucleotide-directed mutagenesis. M13 can be grown on Rec⁻ strains of E. coli. The M13 genome is expandable, and the phage does not lyse cells; rather, the M13 genome is extruded through the membrane and coated by a large number of identical protein molecules. It is therefore possible to insert extra genes into its genome and have them carried along stably.

The M13 major coat protein is encoded by gene VIII. The 50 amino acid mature coat protein is synthesized as a 73 aa precursor, the first 23 aa's of which are a typical signal sequence. An E. coli signal peptidase, SP-I, cuts between residues 23 and 24 of this “precoat.” After removal of the signal sequence, the N-terminus of the mature coat is located on the periplasmic side of the inner membrane; the C-terminus is on the cytoplasmic side. About 3000 copies of the mature, 50 residue long coat protein associate side-by-side in the inner membrane. The amino acid sequence of gene VIII protein can be encoded on a synthetic gene, using the lacUV5 promoter in conjunction with the Lac^{q} repressor. Mature gene VIII protein has only one domain and makes up the sheath around the circular ssDNA.

When M13 phage is used in the present methods, the gene III and gene VIII proteins are highly preferred OSPs. However, the proteins encoded by genes VI, VII, and IX may also be used.

Libraries have been constructed with M13 expressing peptides from 4 to 30 amino acids long with a complexity in the range of 10⁷ to 10¹⁵. (Complexity is a reflection of the number of different sequences expressed, e.g., with 5-mers, the upper limit is 5!; the “complexity” is a fraction 25 of that.)

An M13 combinatorial peptide library expresses random amino acid sequences as fusions with the M13 phage coat protein where they are available to interact with a target protein. For the present method, the “target protein” is the library of proteins or peptides expressed from cDNAs at the surface of the first gDP, preferably T7 phage particles. Members of the second library, e.g., M13 phages expressing a peptide sequence which interacts with the expressed cDNA sequences on the surface of T7, will bind the appropriate immobilized T7 particles.

The two interacting phage types are eluted independently from each pin of the solid (e.g., 96 pin) support. Thus, in the T7-M13 combination, M13 particles can be separated from T7 particles. The DNA of each set of interacting phages is amplified for sequencing using routine PCR methods. The relevant DNA sequences derived from the T7 phage (for the full library), indicate the amino acid sequences of proteins normally expressed in the tissue, organ or organism that was the source of the cDNA library. In contrast, the DNA sequences derived from the M13 library represent amino acid sequences mimicking endogenous proteins that would normally interact with the target proteins expressed on T7.

In a preferred embodiment, the DNA taken from a large number of M13 phage clones (such as about 20, that interacted with the same T7 target population is sequenced, and the nucleotide and encoded amino acid sequences are compared between clones. It is expected that various of the M13 phages will represent overlapping parts of the critical interacting domain; hence, shared, overlapping sequences serve to define the domain. These shared sequences are then compared to an existing database to determine if and how many proteins with such a sequence have been identified. With the imminent completion of the human genome project, it will be quite simple to identify such interacting proteins.

Enhancing the Potential of T7 Phage Display as a Tool for Detection and Assay of Protein-Protein Interactions

The use of T7 as a display vector for tissue specific cDNA libraries may be compromised by the inability to display the putative reactive epitope in a configuration suitable for interaction with protein partners, including antibodies. It is possible that expression of proteins as direct fusions with the 10B capsid protein may sterically interfere with or mask potential interactive domains. To overcome these potential problems, an oligonucleotide spacer encoding a 15 amino acid sequence is inserted at the 5′ cloning site, between the existing 10B cloning site and the expressed cDNA sequence, and flanked by a unique cDNA cloning insertion site at the 3′ end of the spacer. The oligonucleotide preferably encodes a linker (L). A preferred linker is Gly₆Pro₃Gly₆. This sequence has little chance of forming secondary structure with itself or the expressed protein. Those skilled in the art will readily appreciate how to vary this linker for the stated purpose using conventional methods. The presence of this linker will space the expressed protein from the phage surface, allowing more mobility and thus the opportunity for assumption of appropriate secondary configuration. At the same time extension away from the phage surface will allow extended exposure to the aqueous environment.

Negative Selection of Phage T7 Lacking a cDNA Insert

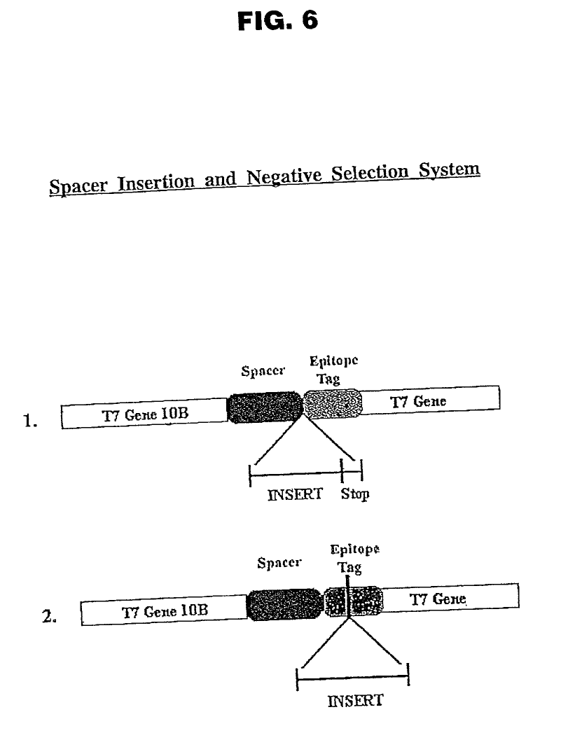

A negative selection system is employed in the construction of phage T7 display libraries (FIG. 6) because the preparation of representative T7 display libraries is invariably accompanied by the recovery of parental phage particles that lack inserts but nevertheless have a certain degree of nonspecific stickiness. Moreover, phage without inserts may overgrow, and lead eventually to the loss of, phage containing inserts. This results from the potential for inserts to compromise phage assembly.

To overcome this problem the present inventors have developed a negative selection system to remove parental phage that lack cDNA inserts. A nucleotide sequence encoding an antibody reactive epitope is inserted at the existing cloning site in the 10B coding sequence such that, when a cDNA insert is absent, the intact antibody epitope is expressed as a fusion with 10B. Phage lacking an insert are selected by an affinity method that removes phage expressing the intact epitope.

Two cloning methods are used to obliterate the antibody epitope:

(1) The cloning site is located between the linker and the epitope. (FIG. 6, top) The cDNA population has a stop codon inserted at the 3′ end such that the antibody epitope is not transcribed in insert-bearing phages. The stop codon is engineered as part of the random primers used to construct the cDNAs and will thus reside at the 3′ end of all clones.

(2) The cloning site is engineered into the oligonucleotide encoding the antibody-reactive epitope such that insertion of cDNAs causes the epitope to be destroyed (FIG. 6, bottom). This is accomplished by identifying key amino acids in that epitope by “alanine scanning.” Once identified, a silent mutation is introduced into the codon for the critical amino acid, at the same time creating a new restriction site useful for cloning. This leaves the amino acid sequence of the immunoreactive epitope intact in the absence of a cDNA insert and destroys the epitope when an insert is present. A preferred negative selection technique involves an epitope of the influenza virus hemagglutinin (HA) protein made up of about 9 amino acid residues. Such a structure is characterized as

Capsid 10B—Linker (L)—HA.

Polyclonal and monoclonal antibodies specific for this epitope are commercially available. The cDNA is inserted either between L and HA or within the HA. It can include a stop codon. If a cDNA insert is present, no HA epitope is formed. HA-bearing phage are selected against as being ones that contain (by definition) no inserts.

As is evident to those skilled in the art, any antibody-recognizable epitope or any binding site for a binding partner can be used for this selective technique.

Other Approaches to Reduce Background Binding

The present inventors have observed that for certain known protein-protein interactions, T7 displaying a protein bound to a binding partner for that displayed protein to a degree comparable to the binding of parent T7 (empty) phage, whether in the presence or absence of calcium ions. Such a background, may also be due to the PBD being in a form in which it cannot easily interact (e.g., steric interference; see above). This can be tested by using an antibody specific for the PBD and comparing its binding of the PBD displayed on T7 OSP to binding of empty T7. One solution to solve this type of background problem is by selection reaction vessel (e.g., microwell) configuration. Flat bottom wells develop a higher surface tension at the “corners.” It is preferred to use modified “flat” V bottom wells that have been designed for ELISA plates and eliminates some background. Another solution involves washing the wells with more force, e.g., using Water-Pik(® device or an equivalent thereof run across plates.

Other Genetic Display Packages

Bacteriophage φX174 as a gDP

φx74 is a very small icosahedral virus which has been thoroughly studied (See Denhardt, DT et al., eds, The Single-Stranded DNA Phages, Cold Spring Harbor Laboratory, 1978). φX174 is not used as a cloning vector because it accepts very little additional DNA (and is so tightly constrained that several of its genes overlap). Three φ174 gene products are on the outside of the mature virion: F (capsid), G (major spike protein, 60 copies per virion, 175 amino acids long), and H (minor spike protein, 12 copies per virion, 328 amino acids long). F interacts with the single-stranded DNA of the virus. F, G, and H (encoded by genes f, g and h, respectively) are translated from a single mRNA in infected cells. If G is supplied from a plasmid in the host, then the viral g gene is no longer essential. For use in this invention, one or more stop codons are introduced into the g gene so that no G is produced from the phage gene. A fragment of a gene encoding the PBD is fused to h, either at the 3′ or 5′ terminus. An amount of the g gene equal to the size of pbd is eliminated so that the size of the genome is unchanged.

Large DNA Phages as gDPs

Phage such as λ or T4 have much larger genomes than do M13 or φX174. Large genomes are less conveniently manipulated than smaller genomes. The genome of λ is so large that cassette mutagenesis is not practicable, and homologous recombination using a mutagenic oligonucleotide cannot be used because there is no ready supply of single-stranded λ DNA (as it is packaged as double-stranded DNA). Phage such as λ and T4 have more complicated 3D capsid structures than M13 or φ174, with more OSPs to choose from. Intracellular morphogenesis of phage λ could prevent protein domains that contain disulfide bonds in their folded forms from folding. Because λ and T4 particles form intracellularly, PBDs requiring large or insoluble prosthetic groups might fold on the surfaces of these phage.

Bacterial Cells as gDPs

One may choose any well-characterized bacterial strain which (1) can be grown in culture 20 (2) can be engineered to display PBDs on its surface, and (3) is compatible with affinity selection methods.

Among bacterial species, those that are preferred as gDPs are Salmonella typhimurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. All bacteria exhibit proteins on their outer surfaces. Descriptions of the localization of OSPs and methods of determining their structure can be found in: von Heijne, G et al., Protein Engineering, 1990, 4:109-112; Lugtenberg, B. et al., Biochim Biophys Acta, 1983, 737:51-115; Silhavy, T J et al., Microbiol Rev, 1985, 49:398-418; Nakae, T, CRC Crit Rev Microbiol, 1986, 13:1-62; Randall, L L et al. Ann Rev Microbiol, 1987, 41:507-41; Manoil, C et al., Topics in Genetics, 1988, 4:223-226; Benz, R, Ann Rev Microbiol, 1988, 42:359-93.

While most bacterial proteins remain in the cytoplasm, others are transported to the periplasmic space or are conveyed and anchored to the outer surface. Still others are exported (secreted) into the medium.

It is well known that DNA encoding the leader or signal peptide from one protein may be attached to the coding DNA of another protein, “protein X,” to form a chimeric gene whose expression causes protein X to appear free in the periplasm . That is, the signal peptide leader causes the chimeric protein to be secreted through the lipid bilayer, after which it is cleaved off by the signal peptidase SP-I in the periplasm.

The use of export-permissive bacterial strains (Liss, L R et al. J Bacteriol, 1985, 164:925-928; Stader, J et al., Genes & Develop, 1989, 3:1045-1052) increases the probability that a signal-sequence-fusion will direct the desired protein or peptide to the cell surface for display. Such strains are preferred.

In E. coli, LamB is a preferred OSP, though E. coli a number of good alternatives can be used in this as well as in other bacterial species. It is possible to systematically determine where to insert a PBD-encoding DNA into an osp gene to obtain display of a PBD on the surface of any bacterium. In view of the extensive knowledge of E. coli, a strain of E. coli, defective in recombination is a preferred candidate as a bacterial gDP.

LamB is a porin for maltose and maltodextrin transport and is also the receptor for adsorption of bacteriophages λ and K10. In the presence of a functional N-terminal sequence, namely; the first 49 amino acids of the mature sequence, LamB is transported to the outer membrane. As with other OSPs, LamB is synthesized with a typical signal-sequence which is removed later. Homology exists between parts of LamB and other E. coli outer membrane proteins OmpC, OmpF, and PhoE, particularly with LamB residues 39-49. The amino acid sequence of LamB is known, and a model has been developed of how it anchors itself to the outer membrane (Benz et al., supra). The location of its maltose-binding and phage binding domains are also known. Using this information, one may identify several strategies by which a library of PBD inserts may be incorporated into lamB to provide a chimeric OSP that displays the PBD on the bacterial outer membrane.

E. coli

LamB has also been expressed in functional form in S. typhimurium, V cholerae, and K. pneumonia, so that one could display a population of PBDs in any of these species as a fusion to E. coli LamB. A maltoporin similar to LamB in K. pneumonia and the D1 protein of P. aeruginosa, (a homologue of E. coli LamB) can be used.

OSP-PBD fusion proteins need not fulfill a structural role in the outer membranes of Gram-negative bacteria because parts of the outer membranes are not highly ordered. For large OSPs there is likely to be one or more sites at which the osp gene can be truncated and fused to pbd gene such that cells expressing the fusion will display PBDs on the cell surface. Fusions of fragments of omp genes with fragments of any gene “X” have led to protein X appearing on the outer membrane (e.g., Charbit, A A et al., Gene, 1988, 70:181-189; Benson, S A et al., Proc Natl Acad Sci USA, 1984, 81:3830-3834). When such fusions have been made, an osp-pbd gene can be designed by substituting pbd sequence for x in the DNA sequence. Otherwise, a useful OSP-PBD fusion can be made and identified by fusing fragments of the best osp DNA to any pbd DNA, expressing the fused gene, and testing the resultant gDPs for display of the PBD, for example using antibodies specific for the PBDs. Spacer DNA encoding flexible linkers, made, e.g., of Gly, Ser, and Asn, may be placed between the osp and pbd sequences to facilitate display. Alternatively, osp DNA is truncated at several sites or in a manner that produces osp fragments of variable length, and the osp fragments are fused to pbd; cells that express the fusion are screened or selected on the basis of their display of PBDs on the cell surface. Another alternative is to include short segments of random DNA in the fusion of osp fragments to pbd and then screen or select the resulting randomly distributed population for members displaying the PBD of interest.

When the PBDs are to be displayed by a chimeric transmembrane protein like LamB, the PBD could be inserted into a loop normally found on the surface portion of LamB. Alternatively, a 5′ segment of the osp gene is fused to the pbd gene fragment; the point of fusion is chosen to correspond to a surface-exposed loop of the OSP and the C-terminal portions of the OSP are omitted. In LamB, up to 60 amino acids may be inserted and result in display of the foreign epitope; the structural features of OmpC, OmpA, OmpF, and PhoE are sufficiently similar to LamB that similar behavior is expected. Thus, other bacterial outer surface proteins, such as OmpA, OmpC, OmpF, PhoE, and pilin, may be used in place of LamB and its homologues. Other bacterial OSPs that could be used for display include E. coli PhoE, BtuB, FepA, FhuA, IutA, FecA, and FhuE.

OmpA is of particular interest because of its great abundance and because knowledge of its homologues in a wide variety of gram-negative species. See Baker, K et al., Prog Biophys Molec Biol, 1987, 49:89-115 for a review of assembly of proteins into the outer membrane of E. coli and describe a model that that predicts that residues 19-32, 62-73, 105-118, and 147-158 are exposed on the cell surface. Insertion of a PBD encoding fragment at about codon 111 or at about codon 152 is likely to cause the PBD to be displayed on the cell surface. Porin Protein F of P. aeruginosa has been cloned and has sequence homology to OmpA of E. coli. OmpF coli is very abundant, ≧10⁴ copies/cell (Pages, J M, Biochimie, 1990, 72:169-176). Fusion of a pbd gene fragment, either as an insert or replacing the 3′ part of ompF, in one of the relevant regions is likely to produce a functional ompF:pbd gene which leads to display of PBD on the bacterial surface.

Pilus proteins are of interest because (a) many copies are expressed on piliated cells and (b) several species (N. gonorrhoeae, P. aeruginosa, Moraxella bovis, Bacteroides nodosus, and E. coli) express related pilins. The N-terminal portions of the pilin protein are highly conserved. Thus a preferred place to attach a PBD (with or without a linker) is the C-terminus.

Protein IA of N. gonorrhoeae has its N-terminus is exposed so that one could attach an PBD at or near the N-terminus of the mature pIA to display the PBD on the N. gonorrhoeae surface.

Bacterial Spores as gDPs

Bacterial spores have desirable properties as gDP candidates. Spores are much more resistant than vegetative bacterial cells or phage to chemical and physical agents, and hence permit the use of a great variety of affinity selection conditions. Bacillus spores neither actively metabolize nor alter the proteins on their surface. Spores have the disadvantage that the molecular mechanisms that trigger sporulation are less well understood than is the life cycle of phage M13 or the export of proteins to the outer membrane of E. coli.

Bacteria of the genus Bacillus form endospores that are extremely resistant to damage by heat, radiation, desiccation and toxic chemicals (reviewed by Losick et al., Ann Rev Genet, 1986, 20:625-669. B. subtilis forms spores in 4 to 6 hours, whereas Streptomyces species may require days or weeks to sporulate. In addition, B. subtilis is much better characterized genetically and is readily manipulated compared to other spore-formers. Viable spores that differ only slightly from wild-type are produced in B. subtilis even if one of four coat proteins is missing. Moreover, plasmid DNA is commonly included in spores, and plasmid encoded proteins have been observed on the spore surface. It should be possible to express during sporulation a gene encoding a chimeric (fused) PBD-coat protein, without interfering materially with spore formation.

Several polypeptide components of B. subtilis spore coat have been identified and the sequences of several complete coat proteins and N-terminal fragments of others are known. Some of the coat proteins are synthesized as precursors and then processed by specific proteases before deposition in the spore coat. The sequence of a mature spore coat protein contains information that causes the protein to be deposited in the spore coat; thus gene fusions that include some or all of a mature coat protein sequence are preferred for the display of PBDs.

The promoter of a spore coat protein is most active when spore coat protein is being synthesized and deposited onto the spore and at the specific place that spore coat proteins are being made. The sequences of several sporulation promoters are known; coding sequences operatively linked to such promoters are expressed only during sporulation. The G4 promoter of B. subtilis is directly controlled by RNA polymerase bound to σ^{E}. The quantity of protein produced from a sporulation promoter can be controlled by factors such as the DNA sequence around the Shine-Dalgarno sequence or by codon usage.

Solid Supports

By “solid support” or “carrier” is intended any support capable of binding a protein (or other ligand material being screened or tested) while permitting washing without dissociating from the ligand. Well-known supports or carriers include, but are not limited to, natural cellulose, modified cellulose such as nitrocellulose, polystyrene, polypropylene, polyethylene, polyvinylidene difluoride, dextran, nylon, polyacrylamide, and agarose or Sepharose®. Also useful are magnetic beads. The support material may have virtually any possible structural configuration so long as the immobilized target peptides or proteins are capable of binding to the PBDs of the ΦDL. Thus, the support configuration can include microparticles, beads, porous and impermeable strips and membranes, the interior surface of a reaction vessel such as test tubes and microtiter plates, and the like. A preferred support is polystyrene in the form of a multiwell microplate. Those skilled in the art will know many other suitable carriers for binding the target peptides will be able to ascertain these by routine experimentation.

Most preferred is a solid support to which the target peptide is attached or fixed by covalent or noncovalent bonds. Preferably, noncovalent attachment is by adsorption using methods that provide for a suitably stable and strong attachment. The peptides are immobilized using methods well-known in the art appropriate to the particular solid support, providing that the ability of the peptides to bind PBDs of the ΦDL is not compromised. For a review of protein immobilization and its use in binding, assays, see, for example, Butler, J. et al. In: Van Regenmortel, ed., Structure of Antigens, Volume 1, CRC Press, Boca Raton, FL, 1992, pp. 209-259. Immobilization may also be indirect, for example by the prior immobilization of a molecule which binds stably to the target peptide or to a chemical entity conjugated to the peptide. For example, an antibody (polyclonal or monoclonal) specific for the target peptide may be immobilized by passive adsorption or covalent attachment. The target peptide is then allowed to bind to the antibody, rendering the peptide immobilized. Indirect immobilization, as intended herein, includes bridging between the peptide and the solid surface using any of a number of well-known agents and systems. For example, the “Protein-Avidin-Biotin-Capture” (PABC) system is described by Suter, M. et al, Immunol. Lett. 13:313-317, 1986). In such a system, any biotinylated protein is immobilized by passive adsorption (or covalent linking) to the solid phase. Streptavidin, which is multivalent, binds with high affinity to the biotin sites on the immobilized protein while maintaining available binding sites for biotin in solution. The target protein or peptide in biotinylated form, is then allowed to bind to the immobilized streptavidin, rendering the target peptide immobile. Alternatively, the streptavidin can be passively adsorbed or covalently bound to the solid phase without the intervening protein. Target peptides immobilized by any of the foregoing approaches (provided that they do not interfere with its ability to bind and retain PBDs is within the scope of the present invention. Any binding partner, such as a protein that binds specifically with the gDP, e.g., an antibody may be immobilized in the foregoing method.

Having now generally described the invention, the same will be more readily understood through reference to the following examples which are provided by way of illustration, and are not intended to be limiting of the present invention, unless specified.

### EXAMPLE I

### Picking Interacting Partners from a T7 Expression Library

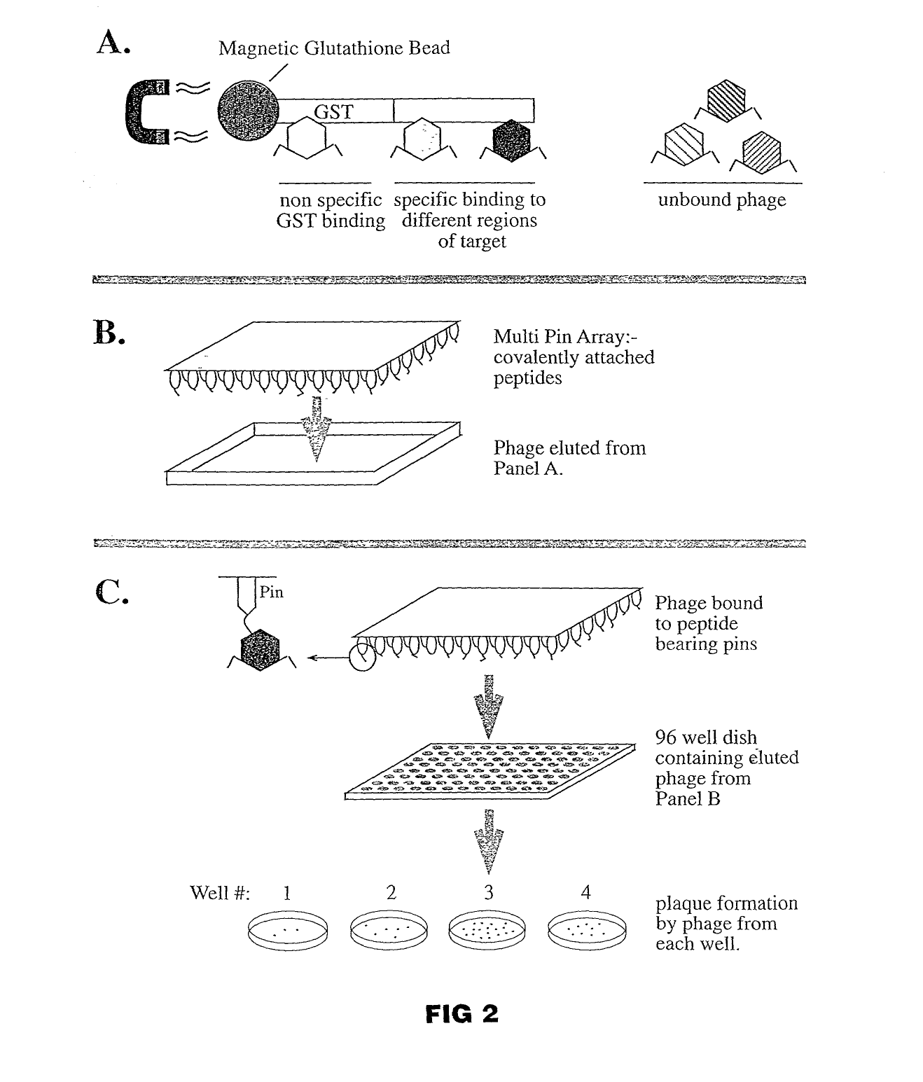

Screening a T7 library is easily accomplished using target proteins or peptides attached to solid state matrices. Initial screen will employ intact proteins, or large regions thereof, attached to magnetic beads. This allows for very rapid and extensive washing in high salt or detergent containing buffers. Proteins will be expressed as fusions with Glutathione-S-Transferase (GST) in E. coli and immobilized on glutathione magnetic beads (FIG. 2A, B, C). The entire phage library is incubated in “batch” with the target protein—such as a GST fusion with the cytoplasmic domain of N-cadherin or P0 attached to the glutathione magnetic beads.

The primary screen, accomplished within several hours, rapidly enriches the pool of phage particles that interact with the target protein. This bound population will contain phage that bind to many distinct regions of the target, as well as some phage that have bound non-specifically to the bead or to GST.

The bound population of phage is eluted, which is extremely simple given the stability of T7, and used immediately for a second screen. Phage expressing sequences that bind to GST or the beads alone are eliminated in the second screen as described below.

### EXAMPLE II

### Second Screen for Phage Recognizing Specific Target Domains

A second screen sorts the phage into populations that recognize specific domains of the target protein. This screen can be completed in the same day as the primary screen.

This is made practical by the recent development of simple and inexpensive peptide synthesis paradigms. Multiple individual peptides are synthesized covalently attached to pins which fit a 96 well microtiter plate. Thus, with little or no mechanization, 96 different peptides can be synthesized simultaneously by addition of the appropriate amino acid to the appropriate well of the 96 well plate (as was described above with citation of relevant references). At the completion of each reaction, the pin bearing the growing amino acid chain is simply removed, washed and transferred to a plate bearing the appropriate distribution of the next amino acid. This system may be expanded to 384 peptides, or multiples thereof, allowing for the simultaneous screening or multiple targets for the phage that display PBDs.

The present inventors use peptides from 10 to 12 amino acids in length as a starting point for producing the target array; for proteins or protein regions of approximately 100 amino acids, it is possible simply to move along the sequence one amino acid at a time, synthesizing overlapping sequences with an offset of one amino acid.

These parameters, of course, are adjustable, but these lengths have been used very effectively in phage display to determine sequences which interact with target proteins (Sparks et al., supra; Kay, B K et al., supra) and as binding partners in direct binding and competition assays (Geysen, H M et al., Proc. Natl. Acad. Sci. USA 81:3998-4002; Geysen et al., 1987, supra); Felder, S. et al., 1993; Mol. Cell. Biol. 13:1449-1455; Case R D et al., 1994, J. Biol. Chem. 269:10467-10474.

This secondary screen not only identifies phage carrying protein segments that interact with specific regions of the target, but helps to identify specific from nonspecific interactions.

If all cDNA fragments were equally represented in the T7 library, we would anticipate that pin bearing target sequences recognized by effector/adaptor molecules will have bound many phage encoding overlapping sets of cDNA sequences (FIG. 2B). In contrast, pin bearing sequences for which there are no interactions will have bound relatively few phage, and these will have non-overlapping sets of sequences reflecting the assay background. In addition, as we move along the pin array representing a protein target, we see increases and decreases in the number of plaques formed by the eluted phage consistent with the distribution of binding domains (FIG. 2C).

It may be that not all cDNAs are equally represented, and some important PBDs may be minimally represented, changing the theoretical distribution of the phage on the target pins. Thus, in defining each new set of targets, it is important to sequence a representative number of phages from all pins.

Critical to the present strategy is the ability to sequence rapidly cDNAs derived from many independent phage isolates. This is readily accomplished using modern equipment such as the ABI 3400 which can sequence 96 samples simultaneously.

### EXAMPLE III

### Synaptotagmin (“Stt”) Interactions

Potentially important targets in nerve synapses for the toxic effects of lead include calcium binding /proteins such as the Synaptotagmins (Syts). Syts I-XI are a family of vesicle proteins that function as calcium sensors to regulate the fusion of neurotransmitter-filled vesicles with the plasma membrane (Sudhof, T C et al., 1996, Neuron 17:379-388.

All Syt isoforms are characterized by an N-terminal intravesicular domain, a single transmembrane domain and a large cytoplasmic region containing two homologous C2 domains (CIA and C2B). Distinct calcium dependent protein interactions involving the C2A and C2B domains of Syts have been proposed to directly regulate neurosecretion. A subset of mutations in the C2B domain of Syt I reduces the calcium responsiveness of neurosecretion (Littleton, J T et al., 1994, Proc. Natl. Acad. Sci. USA 91: 10888-10892). Calcium promotes homo-oligomerization as well as the hetero-oligomerization of Syt I with other isoforms through its C2B domains (Chapman, E R et al., 1998, J. Biol. Chem. 273:32966-32972). The foregoing suggests that oligomer assembly is important for Syt I function in neurosecretion (and because oligomerization is promoted by calcium, lead may target this process and thereby neurosecretion.

Syt IV, a novel member of the Syt family; is an early immediate gene whose expression is rapidly increased during cell depolarization and kainic acid induced epileptic seizures (Vician, L et al., 1995, Proc. Natl. Acad. Sci. USA 92:2164-2168). Syt IV may function with Syt I to regulate neurosecretion (Ferguson G D et al., 1999, J. Neurochem. 72:1821-1831; Thomas D M et al., 1999, Mol Biol. Cell 10:2285-2295; Thomas D M et al., J. Neurosci. 18:3511-3520). Syt IV colocalizes with Syt I on secretory vesicles in neuroendocrine cells. Microinjected recombinant Syt IV fragments blocked calcium stimulated neurotransmitter in neuroendocrine cells.

It is hypothesized that Syt IV regulates neurosecretion by interacting directly with Syt I to alter the calcium sensing properties of the secretory machinery and lead mediates its toxic affects on neurosecretion by directly interfering with the ability of calcium to regulate these interactions.

The present methods permit testing this hypothesis by identifying the amino acids mediating Syt I-Syt IV interactions so that the effects of lead on this specific interaction can be evaluated.

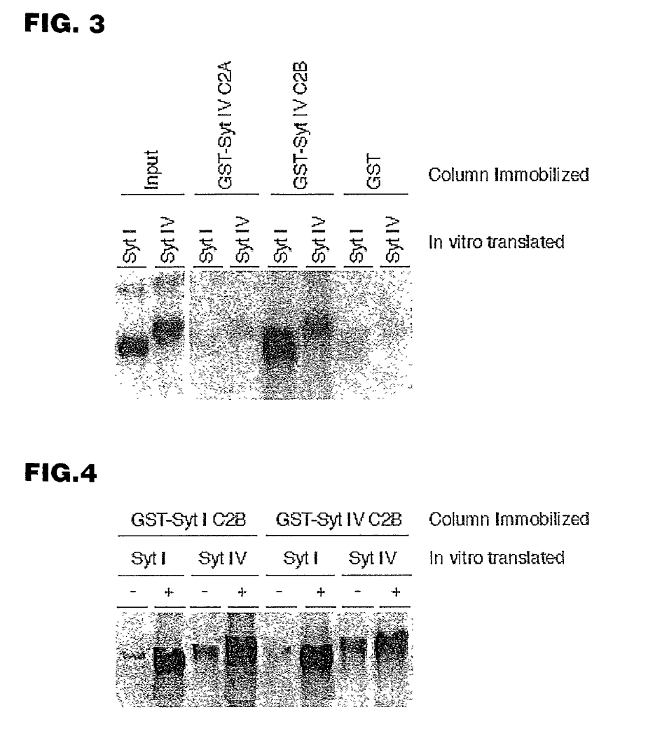

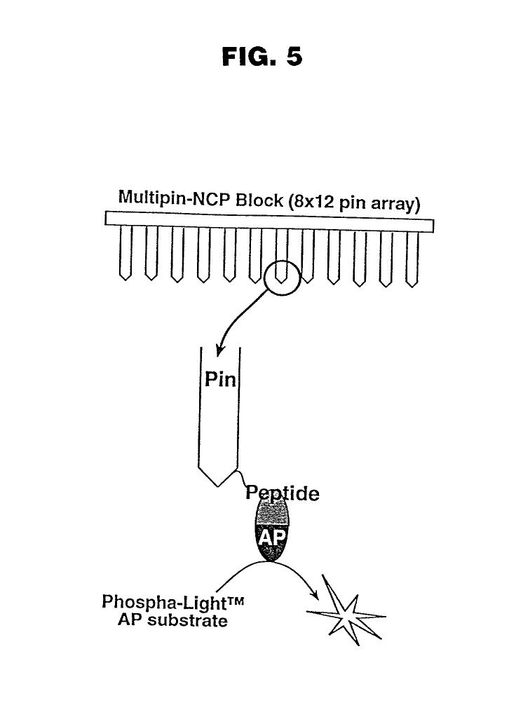

To examine the calcium binding properties of the SytIV C2B domain, we compared the oligomerization properties of Syt IV with Syt I (FIG. 3). The C2A and C2B domains of Syt IV were expressed as GST fusion proteins, immobilized on glutathione agarose and incubated with soluble in vitro translated Syt I or Syt IV. In the presence of calcium, GST alone or the C2A domain of Syt IV show essentially no binding with Syt I or Syt IV (FIG. 3). Conversely, strong Syt I and SytIV binding was observed with the C2B domain of Syt IV. These results indicate that the C2B domain of Syt IV is capable of homo-oligomerization well as hetero-oligomerization with SytI.

To confirm the calcium dependency of these interactions, these studies were performed in the presence or absence of calcium. In the presence of calcium, both immobilized recombinant Syt I and Syt IV C2B domains interact within vitro translated Syt I and Syt IV (FIG. 4). These data indicate that the C2B domain of Syt IV exhibits calcium binding properties which promote both the formation of Syt IV oligomers as well as hetero-oligomers with the C2B domain of Syt I.

Since these 130 amino acid C2B domains are too long for alanine scanning mutagenesis, the inventors use the immobilized peptide assay of this invention to (1) map the interacting amino acid residues and (2) assess the effects of lead in this process.

The successful generation of antibodies against synthetic peptides, epitope mapping, and phage display studies all demonstrate that short peptides can bind to proteins with high affinity and specificity. It is therefore possible to identify the specific amino acid contacts between interacting proteins using peptide-protein interactions.

For practical purposes however, two criteria must be met to render this strategy feasible: Firstly, it is necessary to generate easily, a large number of short peptides (e.g., 6-12 amino acids) that together represent a large portion of a protein, such as a dimerization domain. This criterion is satisfied by the pin synthesis technique devised by Geysen et al. and discussed above, enabling the simultaneous synthesis of as many as 96 individual peptides on polyethylene solid-support pins arranged in an 8-column, 12-row format complementary to a microplate. This multipin peptide synthesis technology is now commercially available from Chiron Mimotopes (Raleigh, N.C.).

Multipin-NCP peptide Synthesis.

All peptide syntheses will use the multipin-NCP (Non Cleavable Peptides) peptide synthesis kits available from Chiron Mimotopes in accordance with he manufacturer's protocol. Briefly, 96-pin blocks provided by the manufacturer contain a t-butyloxycarbonyl (Boc)-protected non-cleavable spacer (Geysen et al., 1987, supra). The pins are initially Boc-deprotected followed by the sequential addition of Fmoc-protected amino acids (Maeji, N J et al., 1990, J. Immunol. Methods. 134:23-33).

At a coupling rate of two residues/pin/day, synthesis of the dodecamer peptides will require six working days. Because individual peptides are synthesized simultaneously, the number of different peptides required is not a limitation. To ensure that the correct amino acid is added to each pin in the array with each cycle in the synthesis, the “PinAID” microcomputer program available from Chiron Mimotopes is employed.

Synaptotagmin-Syntaxin Interactions

This system has both a calcium dependent and a calcium independent interaction which 15 permits demonstration of some of the advantages of the present invention. The present inventors completed a yeast two-hybrid screen using Syt 1, syntaxin 1A and synaptobrevin 2 (Vamp 2). Recombinant and native Syt-1 and syntaxin 1A were shown previously to interact in a calcium dependent manner. Similarly, native and recombinant syntaxin 1A and synaptobrevin 2 were shown to interact directly in a calcium independent. Using the yeast two hybrid system syntaxin 1A and synaptobrevin were found to interact directly, whereas Syt-1 and syntaxin 1A did not. Screens performed using two different approaches—cotransformations and yeast matings—gave identical findings.

The present inventors prepared viable recombinant T7 phage which express these proteins on the virion surface. The cDNAs encoding these proteins range in size from 270-800 bps, indicating that recombinant T7 phage containing large cDNA fragments are viable. These recombinant T7 phage are being used to establish screening conditions for calcium dependent and independent protein-protein interactions.

### EXAMPLE V

### Combining the Power of Phage T7 cDNA Protein Display with M13 Random Peptide Display:

Phage T7 has the capacity to display proteins and protein fragments that are fused to the major capsid protein. Thus using the methods described above, a cDNA library from a biological source is expressed in T7 such that the encoded proteins or peptides are displayed at the phage surface where they are free to interact with protein partners presented in any of a number of different formats.

The approach described above is primarily for screening these T7 cDNA ΦDLs against synthetic peptides representing overlapping segments of predetermined and known proteins of interest. This technology will identify cDNAs encoding binding domains which interact with the target peptides and therefore physiologically or developmentally important signaling intermediates.

In another embodiment, the present approach can be instituted as a general screen for protein-protein interactions when neither binding partner is known. This approach was referred to above as the “double unknown” approach.

A first display library that displays PBDs from a source being screened in a gDP is immobilized. The display library is preferably a ΦDL, and in this example, is a T7 cDNA display library as described above. Immobilization must be done by attaching the gDP though a part of the gDP that will not significantly interfere with display of the PBDs for binding to a second display library. Preferably an antibody to an OSP or other molecular species on the outer surface of the gDP is first immobilized to a solid support. The gDP library is contacted and allowed to bind. In this example, the T7 particles are immobilized via phage tail fibers to a 96 well-format pin apparatus using an antibody specific for the phage tail fiber protein, or an E. coli receptor for this protein, which has been immobilized to each pin. The antibody-coated pins are incubated with T7 phage at an appropriate dilution, resulting in immobilized T7 phage display library.

The pin apparatus with immobilized T7 is then screened against a second combinatorial library displayed in a gDP. This may be a random library, to increase the probability that a cognate binding partner for the immobilized PBDs will be found, selected and identified. In the present example, an M13 phage display combinatorial peptide library is used. However, as described above, any of a number of gDPs can be adapted for this use.

M13 is a filamentous phage, essentially a rod, in contrast to the complex hexagonal structure of T7. Peptides may be expressed as fusions with any of three coat proteins; situated terminally on the rod or distributed about the rod surface. Libraries have been constructed expressing peptides from 4 to 30 amino acids with a complexity of the expressed peptides in the range of 10⁷ to 10¹⁵.

An M13 combinatorial peptide library expresses random amino acid sequences as fusions with the M13 phage coat protein where they are available to interact with a target protein. In this case, the “target protein” is the library of proteins or peptides expressed from cDNAs at the surface of the T7 phage particles. M13 phages expressing a peptide sequence which interacts with the expressed cDNA sequences on the surface of T7 will bind the appropriate immobilized T7 particles.

Phages are independently eluted from each pin of the solid 96 pin support; the M13 particles are separated from the T7 phage, (as described above) and each set of interacting phages is amplified for DNA sequencing.

The DNA sequences derived from the T7 phage represent amino acid sequences of proteins normally expressed in the biological source, e.g., the tissue, organ or organism from which the cDNA library was obtained. In contrast, the DNA sequences derived from M13 represent amino acid sequences mimicking endogenous proteins which would normally interact with the PBDs expressed on T7. In this approach, the distinctions between PBD and target as generally used above become blurred—either library may be considered a library of PBDs and the other can be considered a target library.

In this example, one sequences DNA taken from many (˜20) M13 phage clones that were bound to and eluted from the same T7 target and the nucleotide and encoded amino acid sequences within this group of clones are compared. Shared sequences define the critical interacting domain. These shared sequences are then compared to existing database to determine if and how many proteins with such a sequence have been identified. New interactions will be defined in this manner. Moreover, with the imminent completion of the human genome project, it will be quite simple to identify such interacting proteins from growing databases.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A screening method for identifying, in a library of potential binding domains (PBDs) from a biological source, a polypeptide binding domain or domains that bind to a target epitope or family of target epitopes, comprising:
(a) providing a cDNA library from said source that encodes said library of PBDs as a T7 phage display library wherein the PBDs are displayed on the outer surface of said T7 phages as fusion proteins with an outer surface protein (OSP) of said T7 phages;
(b) contacting said phage display library with a bindable array of target epitopes or families of epitopes under conditions where any of said PBDs binds to said target epitopes;
(c) removing unbound T7 phages from said array of target epitopes, so that phages remaining bound are a first sublibrary enriched for PBD-displaying phages;
(d) eluting bound T7 phage from said array of target epitopes; and
(e) determining the DNA sequence encoding the PBDs from said first sublibrary of eluted T7 phage, thereby identifying the PBDs displayed on said eluted phage by their predicted amino acid sequence.

2. The method of claim 1 wherein at least one of
(i) the PBDs of step (a), or
(ii) the target epitope or family of step (b) are predetermined;

3. The method of claim 2 wherein said target epitope or family of epitopes are predetermined.

4. The method of claim 3 comprising, after said eluting step (d) and before said determining step (e), the step of:
(f) subjecting said eluted phage to at least one additional round of contacting and removing of steps (b) and (c) to further enrich phage displaying said PBDs that bind to set predetermined target epitope or epitopes, thereby obtaining a second sublibrary and subsequent sublibraries.

5. The method of claim 4 wherein step (f) is repeated more than once prior to said determining step (e), after each repeat obtaining a new subsequent sublibrary.

6. The method of any of claims 1-5 wherein said outer surface protein capsid protein encoded by gene 10A or 10B of phage T7.

7. The method of claim 6 wherein said outer surface protein capsid protein encoded by gene 10B of phage T7.

8. The method of claim 7 wherein in said display library, said PBDs are expressed in a copy number of about 5-10 PBDs per phage particle.

9. The method of claim 7 wherein, in said phage display library, said PBDs are expressed in high copy number of 415 PBDs per page particle.

10. The method of claim 7 wherein in said phage display library, said PBDs are expressed in an intermediate copy number of about 100 to about 150 PBDs per page particle.

11. The method of any of claims 1-5, wherein said determining step (e) is performed by plating said eluted phage on a lawn of E. coli, permitting them to multiply and form plaques, and sequencing the DNA of the phages of any given plaque to obtain the sequence of the cDNA insert that encodes said PBD.

12. The method of any of claims 1-5, wherein said target epitopes are peptide epitopes and said family comprises peptides or polypeptides corresponding to (i) a protein fragment, (ii) a protein domain or (iii) a complete protein.

13. The method of claim 12, wherein said family of target peptide epitopes comprises a progressive series of overlapping peptides of about 10 to 15 amino acids, each of which peptides lacks n amino-terminal amino acid residues of its predecessor peptide in the series and has at least n additional amino acids added to its carboxy-terminus, wherein n is an integer between 1 and 5, and wherein said series of overlapping peptides corresponds to (i) a region of said protein of up to about 100 amino acids, or (ii) said complete protein.

14. The method of claim 12 wherein said target peptides are synthesized in parallel on polyethylene pins mounted on blocks which are compatible with standard microplate arrays of 96 wells or multiples thereof.

15. The method of claim 13 wherein said target peptides are synthesized in parallel on polyethylene pins mounted on blocks which are compatible with standard microplate arrays of 96 wells or multiples thereof.

16. The method of claim 14, wherein the target peptides are covalently attached to the pins so that said, after said eluting of said bound phages, the blocks are reused for one or more additional screening assays.

17. The method of claim 15, wherein the target peptides are covalently attached to the pins so that said, after said eluting of said bound phages, the blocks are reused for one or more additional screening assays.

18. The method of claim 17, wherein the target peptides are in a cleavable form, allowing recovery of said peptides.

19. The method of any of claims 1-5, wherein said cDNA library is produced from mRNA molecules of said biological source by random priming wherein each cDNA molecule reverse transcribed from said mRNA molecules is between about 50 and about 5000 bp in length, the cDNA molecules are gel purified and directionally cloned into said T7 phage DNA resulting in fused DNA, and said fused DNA is packaged into phage in vitro.

20. The method of claim 19 wherein the cDNA molecule is between about 50 and about 1000 bp in length.

21. The method of claim 20 wherein the cDNA molecule is between about 50 and 500 bp in length.

22. The method of claim 21 wherein the cDNA molecule is between about 100 and 200 bp in length.

23. A method to determine the representation of expressed sequences in a PBD display sublibrary, when said PBDs are from a known protein and specific antibodies for epitopes of the known protein are available,
(i) providing a collection of antibodies specific for the epitopes of the known protein which antibodies are immobilized to a solid support;
(ii) carrying out the method of claim 5 or 6 up to an eluting step wherein the first sublibrary, the second sublibrary or a subsequent sublibrary is obtained;
(iii) contacting the sublibrary obtained in step (ii) with the antibodies of step (i) and permitting the antibodies to bind to the epitopes of the displayed PBDs
(iv) evaluating the results of the binding, thereby determining the representation of the expressed sequences in said sublibrary.

24. The method of claim 23, wherein the solid support is magnetic beads.

25. The method of claim 23, comprising, in addition to the antibody binding steps, the step of obtaining multiple separate phage clones from the sublibrary, separately isolating the DNA therefrom, and sequencing the cDNA insert of each clone that encodes the PBD of that clone.

26. The method of any of claims 1-5 wherein the biological source is selected from the group consisting of developing chick neural retina, cultured neonatal rat Schwann cells, and -myelinating sciatic nerves of 15-25 day old rat.

27. The method of claim 26 wherein the biological source is the Schwann cells or the sciatic nerves, and the target epitopes are peptides of a peripheral myelin protein selected from the group of proteins consisting of PMP22, P0, connexin 32 and EGR2.

28. The method of claim 26, wherein the target epitopes are peptides from the cytoplasmic domain of peripheral myelin protein P0.

29. The method of any of claims 1-5, wherein
(a) the phage display library displays PBDs of a protein selected from the group consisting of β-catenin, PTP1B, p120ctn and Shc; and
(b) the target epitopes are peptides of N-cadherin.

30. The method of any of claims 1-5, wherein
(a) the phage display library displays PBDs of synaptotagmin SytI and the target epitopes are peptides of synaptotagmin Syt IV; or
(b) the phage display library displays PBDs of SytIV and the target epitopes are peptides of Syt I.

31. The method of any of claims 1-5, wherein
(a) the phage display library displays PBDs of SytI or Syt IV and the target epitopes are peptides of syntaxin; or
(b) the phage display library displays PBDs of syntaxin and the target epitopes are peptides of Syt I or Syt IV.

32. A method of identifying peptides participating in protein-protein interactions by screening a first peptide display library for members that interact with a second peptide display library, the method comprising
(a) providing a first cDNA library from a biological source that encodes PBDs as a first T7 phage display library wherein the PBDs are displayed on the outer surface of said T7 phages as fusion proteins with an outer surface protein of said T7 phages, which first display library is immobilized to a solid support and said PBDs are available for binding to a peptide for which they have binding specificity;
(b) providing the second library which is a combinatorial library of peptides displayed on genetic display packages other than T7 that are available for binding to the immobilized members of said first library;
(c) contacting the members of said immobilized T7 first library with members of said second library;
(d) removing unbound particles of both of said libraries so that second library particles remaining bound are enriched for those displaying peptides that bind to the PBDs displayed on the T7 phages,
(e) eluting the bound particles
(f) selectively growing the T7 phages and said genetic display packages under conditions wherein either the T7 phages or the genetic display packages have a growth advantage to obtain enriched populations of the T7 phages expressing said first library and the genetic display packages expressing said second library;
(g) separately amplifying the DNA of the second library particles and the immobilized first library phages to which the second library particles had been bound, and sequencing amplified DNA libraries, thereby determining the predicted amino acid sequences of
(i) the PBDs normally expressed in the biological source that participate in said protein-protein interactions with said second library peptides, and
(ii) the peptides that are part of, or that mimic, endogenous proteins that normally interact with said first library PBDs
thereby identifying the peptides participating in the protein-protein interactions

33. The method of claim 32, wherein immobilization is by an antibody specific for an outer surface structure of said T7 phage

34. The method of claim 33, wherein said outer surface structure is a tail fiber.

35. The method of claim 32 wherein said genetic display package is a phage.

36. The method of claim 35 wherein the phage is M13.

37. The method of claim 36 wherein the second library is an M13 random combinatorial peptide library.

38. The method of claim 37 wherein members of said second library have from about 4 to about 30 amino acids with a complexity of expressed peptides of between about 10⁷ and about 10¹⁵.
